(19) United States
(12) Patent Application Publication
(10) Pub. No.: US 2025/0000043 A1
(43) Pub. Date: Jan. 2, 2025

(54) NON-TRANSGENIC SUNFLOWER PLANTS HAVING INCREASED TOLERANCE TO HERBICIDES

(71) Applicant: BASF Agricultural Solutions Seed US LLC, Florham Park, NJ (US)

(72) Inventors: Bianca Assis Barbosa MARTINS, Limburgerhof (DE); Stefan JANSENS, Gent (BE)

(21) Appl. No.: 18/696,265

(22) PCT Filed: Sep. 26, 2022
(86) PCT No.: PCT/US2022/077037

§ 371 Date: Mar. 27, 2024

(30) Foreign Application Priority Data
Sep. 27, 2021 (EP) 21199213.6
Jul. 7, 2022 (EP) 22183513.5

(51) Int. Cl. A01H 6/14; A01H 1/00; A01H 1/06; C12N 9/02

(52) U.S. Cl. CPC A01H 6/1464; A01H 1/06; A01H 1/123; C12N 9/001; C12Y 103/03004

(57) ABSTRACT

The present invention relates to a non-transgenic sunflower plant comprising a mutated protoporphyrinogen IX oxidase (PPO) gene encoding a mutated sunflower protoporphyrinogen IX oxidase. The present invention further relates to a method of weed control at a plant cultivation site, comprising providing the plant of the present invention and applying to said site an effective amount of a PPO inhibiting herbicide. Further encompassed by the present invention is a method for producing sunflower oil.

### CROSS REFERENCE TO RELATED APPLICATIONS

This application claims the benefit of European Application No. 22183513.5, filed Jul. 7, 2022 and European Application No. 21199213.6, filed Sep. 27, 2021, the contents of which are hereby incorporated herein in their entirety by reference.

### FIELD OF THE INVENTION

The present invention relates to a non-transgenic sunflower plant comprising a mutated protoporphyrinogen IX oxidase (PPO) gene encoding a mutated sunflower protoporphyrinogen IX oxidase. The present invention further relates to a method of weed control at a plant cultivation site, comprising providing the plant of the present invention and applying to said site an effective amount of a PPO inhibiting herbicide. Further encompassed by the present invention is a method for producing sunflower oil.

### BACKGROUND

Herbicides that inhibit protoporphyrinogen oxidase (hereinafter referred to as Protox or PPO; EC 1.3.3.4), a key enzyme in the biosynthesis of protoporphyrin IX, have been used for selective weed control since the 1960s. PPO catalyzes the last common step in chlorophyll and heme biosynthesis which is the oxidation of protoporphyrinogen IX to protoporphyrin IX.

PPO-inhibiting herbicides include many different structural classes of molecules (Duke et al. 1991. Weed Sci. 39:465; Nandihalli et al. 1992. Pesticide Biochem. Physiol. 43:193; Matringe et al. 1989. FEBS Lett. 245:35; Yanase and Andoh. 1989. Pesticide Biochem. Physiol. 35:70). These herbicidal compounds include the diphenylethers {e.g. lactofen, (+−)-2-ethoxy-1-methyl-2-oxoethyl 5-{2-chloro-4-(trifluoromethyl) phenoxy}-2-nitrobenzoate; acifluorfen, 5-{2-chloro-4-(trifluoromethyl) phenoxy}-2-nitrobenzoic acid; its methyl ester; or oxyfluorfen, 2-chloro-1-(3-ethoxy-4-nitrophenoxy)-4-(trifluorobenzene)}, oxidiazoles, (e.g. oxidiazon, 3-{2,4-dichloro-5-(1-methylethoxy)phenyl}-5-(1,1-dimethylethyl)-1,3,4-oxadiazol-2-(3H)-one), cyclic imides (e.g. S-23142, N-(4-chloro-2-fluoro-5-propargyloxyphenyl)-3,4,5,6-tetrahydrophthalimide; chlorophthalim, N-(4-chlorophenyl)-3,4,5,6-tetrahydrophthalimide), phenyl pyrazoles (e.g. TNPP-ethyl, ethyl 2-{1-(2,3,4-trichlorophenyl)-4-nitropyrazolyl-5-oxy}propionate; M&B 39279), pyridine derivatives (e.g. LS 82-556), and phenopylate and its O-phenylpyrrolidino- and piperidinocarbamate analogs. Many of these compounds competitively inhibit the normal reaction catalyzed by the enzyme, apparently acting as substrate analogs. Application of PPO-inhibiting herbicides results in the accumulation of protoporphyrinogen IX in the chloroplast and mitochondria, which is believed to leak into the cytosol where it is oxidized by a peroxidase. When exposed to light, protoporphyrin IX causes formation of singlet oxygen in the cytosol and the formation of other reactive oxygen species, which can cause lipid peroxidation and membrane disruption leading to rapid cell death (Lee et al. 1993. Plant Physiol. 102:881). Not all PPO enzymes are sensitive to herbicides which inhibit plant PPO enzymes. Both the Escherichia coli and Bacillus subtilis PPO enzymes (Sasarmen et al. 1993. Can. J. Microbiol. 39:1155; Dailey et al. 1994. J. Biol. Chem. 269:813) are resistant to these herbicidal inhibitors. Mutants of the unicellular alga Chlamydomonas reinhardtii resistant to the phenylimide herbicide S-23142 have been reported (Kataoka et al. 1990. J. Pesticide Sci. 15:449; Shibata et al. 1992. In Research in Photosynthesis, Vol. III, N. Murata, ed. Kluwer: Netherlands. pp. 567-70). At least one of these mutants appears to have an altered PPO activity that is resistant not only to the herbicidal inhibitor on which the mutant was selected, but also to other classes of protox inhibitors (Oshio et al. 1993. Z. Naturforsch. 48c: 339; Sato et al. 1994. In ACS Symposium on Porphyric Pesticides, S. Duke, ed. ACS Press: Washington, D.C.).

A mutant tobacco cell line has also been reported that is resistant to the inhibitor S-21432 (Che et al. 1993. Z. Naturforsch. 48c: 350). Auxotrophic E. coli mutants have been used to confirm the herbicide resistance of cloned plant PPO-inhibiting herbicides. Three main strategies are available for making plants tolerant to herbicides, i.e. (1) detoxifying the herbicide with an enzyme which transforms the herbicide, or its active metabolite, into non-toxic products, such as, for example, the enzymes for tolerance to bromoxynil or to glufosinate (Basta®); (2) mutating the target enzyme into a functional enzyme which is less sensitive to the herbicide, or to its active metabolite, such as, for example, the enzymes for tolerance to glyphosate (Padgette S. R. et al., J. Biol. Chem., 266, 33, 1991); or (3) overexpressing the sensitive enzyme so as to produce quantities of the target enzyme in the plant which are sufficient in relation to the herbicide, in view of the kinetic constants of this enzyme, so as to have enough of the functional enzyme available despite the presence of its inhibitor. The third strategy was described for successfully obtaining plants which were tolerant to PPO inhibitors (see e.g. U.S. Pat. No. 5,767,373 or U.S. Pat. No. 5,939,602). In addition, US 2010/0100988 and WO 2007/024739 disclose nucleotide sequences encoding amino acid sequences having enzymatic activity such that the amino acid sequences are resistant to PPO inhibitor herbicidal chemicals.

WO 2012/080975 discloses plants the tolerance of which to a PPO-inhibiting herbicide named “benzoxazinone-derivative” herbicide (1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione) had been increased by transforming said plants with nucleic acids encoding mutated PPO mutated enzymes. In particular, WO 2012/080975 discloses that the introduction of nucleic acids which code for a mutated PPO of an Amaranthus type II PPO in which the Arginine at position 128 had been replaced by a leucine, alanine, or valine, and the phenylalanine at position 420 had been replaced by a methionine, cysteine, isoleucine, leucine, or threonine, confers increased tolerance/resistance to a benzoxazinone-derivative herbicide. Amongst other crops, sunflower is mentioned as a target crop.

Furthermore, WO 2013/189984 discloses that the introduction of nucleic acids which code for a mutated PPO having a substitution corresponding to the Leucine at position 397, and a substitution corresponding to the phenylalanine at position 420 in the Amaranthus type II PPO, confers increased tolerance/resistance to a variety of PPO inhibitors herbicide. Amongst other crops, sunflower is mentioned as a target crop.

Furthermore, WO2015/022636 discloses substitutions for R¹²⁸, and F420 corresponding to the Amaranthus type II PPO. Amongst other crops, sunflower is mentioned as a target crop.

WO 2016/203377 A1 discloses substitutions for R¹²⁸, L397 and F420 corresponding to the Amaranthus type II PPO.

In general, plants contain two nuclear encoded genes (PPO type I and PPO type II) producing isoforms of this enzyme. PPO1 is compartmentalized in the chloroplast and PPO2 is compartmentalized in the mitochondria, respectively, meaning that PPO inhibitors have two herbicide target sites in plants (i.e., plastids and mitochondria; Jacobs, J. M. & Jacobs, N. J. (1984) Arch. Biochem. Biophys. 229, 312-319). Therefore, in order for target-site resistance to occur, both PP01 and PPO2-altered genes would need to be selected. However, A. tuberculatus plants have overcome this obstacle by means of mutation in a single gene (PPO type II) that is predicted to encode both plastidic and mitochondrial PPO isoforms (Patzoldt W L, Hager A G, McCormick J S and Tranel P J, A codon deletion confers resistance to herbicides inhibiting protoporphyrinogen oxidase. Proc Natl Acad Sci USA 103:12329-12334 (2006).

The dual targeting of PPO Type II in A. tuberculatus is proposed to be caused by the use of two in-frame start codons that create two different length proteins, identical to the mechanism found in spinach (Spinacia oleracea) (Watanabe N, Che F-S, Iwano M, Takayama S and Yoshida S, Dual targeting of spinach protoporphyrinogen oxidase II to mitochondria and chloroplasts by alternative use of two in-frame initiation codons. J Biol Chem 276:20474-20481 (2001). The approximately 30 amino acid extension resulting from a translation initiation at the first start codon encodes a chloroplastic-targeting sequence, resulting in a larger protein being targeted to the chloroplast than that targeted to the mitochondria. Amaranthus tuberculatus had a similar upstream start codon, and the sequence following was identified as a chloroplastic-targeting peptide, (Patzoldt WL, Hager AG, McCormick J S and Tranel PJ, A codon deletion confers resistance to herbicides inhibiting protoporphyrinogen oxidase. Proc Natl Acad Sci USA 103:12329-12334 (2006).

Consequently, previous transgenic approaches for the creation of PPO tolerant plants relying on the Amaranthus PPO type II gene, predicted to encode both plastidic and mitochondrial isoforms by dual targeting (see above) should have led to the expression of resistant PPO enzymes in both compartments.

Li et al. (Plant Physiol 133:736-747 (2003) worked on the development of the mutated PPO as a selection marker in maize and concluded that a given mutation frequently does not behave identically when inserted into a different plant enzyme. The strong correlation of resistant mutations with compromised enzyme activity tends to make many mutation/gene combinations poor candidates for engineering crop tolerance.

Herbicides targeting PPO have a very rapid contact action, causing leaf burning, desiccation and growth inhibition (Li and Nicholl, Development of PPO inhibitor-resistant cultures and crops. PestManag Sci 61:277-285 (2005)). Although PPO targeting herbicides were developed more than 50 years ago, natural occurrence of weed resistance to PPO inhibitors has only been reported for a few plants, for example for Amaranthus palmeri (Salas et al Manag Sci. 2016 May; 72 (5): 864-9. doi: 10.1002/ps.4241. Epub 2016 Mar. 4. PMID: 26817647; PMCID: PMC5069602.

Li and Nicholl (supra) describe that PPO herbicide-resistance mutations tend to reduce enzymatic function. This could explain why only a few plants developed resistant enzymes so far.

Li et al. (2003, supra) describe that it was impossible to develop a field-resistant transgenic maize event without an increase in promoter activity driving the mutant PPO gene.

In WO 2012/080975, WO2015/022636 and WO 2016/203377 (cited above) transgenic plants have been produced expressing mutated PPO genes under control of the ubiquitin promoter which is a strong constitutive promoter.

Sunflower (Helianthus annuus) is an important crop plant that is grown worldwide in temperate and subtropical climates. Sunflower is used primarily for the production of vegetable oil. Sunflower seeds are also used for animal feed (such as bird feed) and food manufacture.

The modification of the genome of sunflower is associated with some drawbacks. Gene editing techniques can be difficult to apply in sunflower breeding, mainly due to the difficulties that occur during plant regeneration and low numbers of obtained transgenic regenerants per assay. Thus, the use of the modern gene editing techniques would require the establishment of improved basis for transformation, which could be beneficial in the development of broomrape resistance in sunflower (Genetic and Genomic Tools in Sunflower Breeding for Broomrape Genes 2020, Resistance 11, 152; doi:10.3390/genes11020152).

The genome from sunflower has been sequenced. It is known that sunflower comprises two PPO genes, PPO1 and PPO2. For example, the sequence of the sunflower PPO2 gene is disclosed under NCBI-Protein ID XP_021982414.1. However, the PPO genes have not been analyzed so far, for example, in the context of PPO tolerance.

Sunflower plants are dicotyledonous plants. It has been described that PPO herbicides are generally more active on dicots than on monocots (Witkowski DA and Halling BP, Inhibition of plant protoporphyrinogen oxidase by the herbicide acifluorfen-methyl. Plant Physiol 90:1239-1242 (1989). Thus, dicotyledonous plants are very sensitive to PPO herbicides. This has been confirmed in the studies underlying the present invention which show conventional sunflowers are highly susceptible to the herbicide saflufenacil (Kixor™), even at very low concentrations. Control of all sunflower materials was around 80% at the lowest rate of saflufenacil (Kixor™, 0.91 g a.i./ha) and 100% at 1.75 g a.i./ha and higher (see Example 1). The high susceptibility of sunflower to PPO herbicides makes it challenging to identify sunflowers that have sufficient levels of herbicide tolerance PPO inhibiting herbicides in general.

Currently, PPO herbicides are used for sunflower in pre-plant burndown and pre-emergence applications for controlling weeds, i.e. before the emergence of the sunflower plant. Therefore, there is a strong need to provide sunflower plants, which show tolerance to PPO herbicides, applied in post-emergence. Such plants allow treatment with PPO herbicides at a later stage of the growing season.

To our knowledge, the prior art has yet not described non-transgenic sunflower plants, which are tolerant/resistant to a broad spectrum of PPO inhibitors. Therefore, what is needed in the art are non-transgenic sunflower plants having increased tolerance to PPO-inhibiting herbicides. Also needed are methods for controlling weed growth in the vicinity of such crop plants or crop plants, in particular post-emergence. These methods would allow for the use of spray over techniques when applying PPO herbicides to areas containing the non-transgenic sunflower plants.

### FIGURE LEGENDS

FIG. 1: Phytotoxicity (%) at 14 days after treatment across increasing saflufenacil (Kixor™) rates-field trial 1.

FIG. 2: Phytotoxicity (%) at 14 days after treatment across increasing saflufenacil (Kixor™) rates-field trial 2.

FIG. 3: Tolerance efficacy (%) of the three PPO2_F383I mutant entries at 2, 5, 12, 19 and 26 days after saflufenacil treatment in the field. Plant recovery is observed as soon as 19 days after treatment (DAT). Utrera, Spain, 2021.

FIG. 4: Phenotypic response of untreated and treated plants of the wild type susceptible reference and mutant entries PPO2_F383I mutant line 1, PPO2_F383I mutant line 2 and PPO2_F383I mutant line 3 at 19 days after saflufenacil treatment. Clear plant recovery is observed after 19 days.

FIG. 5: Amino acid sequence of the mutated sunflower PPO2 (SEQ ID NO: 2). The mutation (F383I substitution) at position 383 is indicated by black shading.

FIG. 6: Protein encoding nucleotide sequence (CDS) of the mutated sunflower PPO2 (SEQ ID NO: 1). The codon, encoding amino acid residue 383 in the PPO2 protein, is indicated by black shading and is given in upper case letters. The mutated sunflower PPO2 shows a T to A transition (TTT to ATT) in the first base of this codon when compared to wild type, resulting in the F383I substitution in the mutated PPO2 protein.

FIG. 7: First N-terminal 50 amino acids (SEQ ID NO: 6) of the Amaranthus tuberculatus PPO2 compared to the first N-terminal 20 amino acids (SEQ ID NO: 7) of the Helianthus annuus PPO2.

FIG. 8: Visual appearance of treated PPO2_F4201 and WT plants across herbicide treatments at 10 DAT: A) with saflufenacil, B) with flumioxazin, C) with saflufenacil and trifludimoxazin, D) with methyl 2-[2-[2-bromo-4-fluoro-5-[3-methyl-2,6-dioxo-4-(trifluoromethyl)pyrimidin-1-yl]phenoxy]phenoxy]-2-methoxy-acetate.

FIG. 9: Proportion of plants at eleven phytotoxicity categories (0-100%) across herbicide treatments for PPO2_F4201 mutants (‘MUTANT’) and wild type line (‘WT’). Phytotoxicity across treated plants for every herbicide treatment is shown along each row of the table. The plants were treated with saflufenacil (800), with flumioxazin (9155), C) with saflufenacil and trifludimoxazin (851), and with methyl 2-[2-[2-bromo-4-fluoro-5-[3-methyl-2,6-dioxo-4-(trifluoromethyl)pyrimidin-1-yl]phenoxy]phenoxy]-2-methoxy-acetate (201).

### BRIEF SUMMARY OF THE PRESENT INVENTION

The present invention relates to a non-transgenic sunflower plant comprising a mutated protoporphyrinogen IX oxidase (PPO) gene encoding a mutated sunflower protoporphyrinogen IX oxidase, wherein the mutated sunflower protoporphyrinogen IX oxidase comprises a substitution of phenylalanine (F) to isoleucine (I) at a position corresponding to residue 383 (F383I substitution).

In an embodiment of the present invention, the non-transgenic plant has not been exclusively obtained by means of an essentially biological process. Thus, the plant shall have been obtained by means other than exclusively an essentially biological process.

In an embodiment of the present invention, the mutated protoporphyrinogen IX oxidase comprises: an amino acid sequence as shown in SEQ ID NO: 2, or a is a variant thereof being at least 98%, such as at least 99% or at least 99.5% identical to SEQ ID NO: 2, with the proviso that the variant comprises a substitution of phenylalanine (F) to isoleucine (I) at a position corresponding to residue 383.

In an embodiment of the present invention, the mutated protoporphyrinogen IX oxidase (PPO) gene comprises
a) a nucleic acid sequence as shown in SEQ ID NO: 1, or
b) a nucleic acid sequence being at least 98%, such as at least 99% or at least 99.5% identical to SEQ ID NO: 1.

In an embodiment, the mutated protoporphyrinogen IX oxidase (PPO) gene is the mutated protoporphyrinogen IX oxidase (PPO) gene of the sunflower plant obtained from growing a seed of mutant line 21LHHA000892, a sample of said seed having been deposited under NCIMB accession number 43974.

In an embodiment of the present invention, the plant is resistant to one or more PPO-inhibiting herbicides.

In an embodiment of the present invention, the mutated PPO gene is present in homozygous form in the plant.

In an embodiment of the present invention, the PPO gene has been mutated by EMS (ethyl methanesulfonate) mutagenesis.

In an embodiment of the present invention, the PPO gene has been mutated by radiation induced mutagenesis.

In an embodiment of the present invention, the PPO gene has been mutated by genome editing.

In an embodiment of the present invention, the non-transgenic sunflower plant comprises on its leaves an effective amount of one or more PPO-inhibiting herbicides.

In an embodiment of the present invention, the PPO gene is operably linked to the native promoter of the protoporphyrinogen IX oxidase (PPO) gene.

In an embodiment of the present invention, the PPO gene is operably linked to the native promoter of the protoporphyrinogen IX oxidase (PPO) type II gene.

In an embodiment of the present invention, the PPO gene contains the nucleotide triplet ATT coding for isoleucine at the position corresponding to amino acid residue 383.

In an embodiment of the present invention, the one or more PPO-inhibiting herbicides are selected from the group consisting of the PPO-inhibiting herbicides shown in Table A. (and combinations thereof)

In some embodiments of the present invention, the one or more PPO-inhibiting herbicide is (are) carfentrazone-ethyl, flumioxazin, saflufenacil and/or trifludimoxazin and combinations thereof.

In an embodiment of the present invention, the PPO-herbicide is saflufenacil.

In an embodiment of the present invention, the non-transgenic sunflower plant comprises a phenotype of tolerance to saflufenacil that is greater than 80% tolerance to 5 g a.i./ha saflufenacil if applied at the 2-to-4 leaf-stage.

In an embodiment of the plant of the present invention, the plant possesses a phenotype of tolerance to one or more PPO-inhibiting herbicides (such as to saflufenacil), which tolerance is greater than that of a corresponding wild-type sunflower plant.

In an embodiment of the present invention, the non-transgenic sunflower plant is obtained from growing a seed of mutant line 21LHHA000892, a sample of said seed having been deposited under NCIMB accession number 43974. Also, the non-transgenic sunflower plant may be a progeny of said non-transgenic sunflower plant, wherein said progeny comprises the mutated protoporphyrinogen IX oxidase (PPO) gene.

Preferably, the non-transgenic sunflower plant or progeny thereof comprises a mutated protoporphyrinogen IX oxidase (PPO) gene comprising a nucleic acid sequence as shown in SEQ ID NO: 1.

The present invention further relates to a seed from the non-transgenic sunflower plant of the present invention, wherein said seed comprises the mutated protoporphyrinogen IX oxidase (PPO) gene. In an embodiment, the seed has on its surface an effective amount of one or more PPO-inhibiting herbicides.

In an embodiment, the seed is a seed of the sunflower line designated “21LHHA000892”, a sample of said seed having been deposited under NCIMB accession number 43974.

The present invention further relates to a method for weed control at a plant cultivation site, comprising the steps of
a) providing a non-transgenic sunflower plant of the present invention at said plant cultivation site, and
b) applying an effective amount of one or more PPO-inhibiting herbicides at said site.

The present invention also relates to a method for treating a plant, comprising the steps of
a) providing a non-transgenic sunflower plant of the present invention, and
b) applying an effective amount of one or more PPO-inhibiting herbicides to said plant.

The present invention further relates to a method for producing sunflower oil, comprising
a) growing the non-transgenic sunflower plant of the present invention at a plant cultivation site,
b) harvesting seeds from the plant, and
c) extracting sunflower oil from the seeds harvested in step b.

In an embodiment of the methods of the present invention, the methods comprise the application of an effective amount of one or more PPO-inhibiting herbicides at the cultivation site, or to said plant.

In an embodiment of the methods of the present invention, the effective amount one or more PPO-inhibiting herbicides is an amount which is capable of controlling a weed, such as a weed of the genus Helianthus, Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and/or Apera.

The present invention further relates to a method for producing sunflower oil, comprising
a) providing a seed of the present invention, and
b) extracting sunflower oil from the seed.

Further encompassed by the present invention is a method for identifying a sunflower plant having improved resistance to one or more PPO-inhibiting herbicides, comprising
a) providing the seed of the present invention or a cell from the non-transgenic sunflower plant of the present invention,
b) subjecting said seed or cell to mutagenesis or transgenesis,
c) growing a plant from said seed or regenerating a plant from said cell, and
d) contacting the plant or a progeny thereof with one or more PPO-inhibiting herbicides, and
e) identifying a plant having improved resistance to one or more PPO-inhibiting herbicides.

In an embodiment of the above method, the mutagenesis step is a step of random mutagenesis that may be achieved via tissue culture, chemical or physical mutagenesis. In an embodiment, mutagenesis is achieved by chemical or physical mutagenesis, e.g. of the seeds. The chemical and physical mutagenesis steps may employ the use of chemical and physical mutagens, such as EMS and ionizing radiation, respectively,

In an embodiment of the above method, the mutagenesis step is based on genome editing.

The present invention further relates to a method for identifying and/or selecting a sunflower plant or seed having resistance to one or more PPO-inhibiting herbicides, comprising
a) providing a biological sample from a non-transgenic sunflower plant of the present invention or from the seed of the present invention,
b) identifying or detecting in said sample the presence of a mutated protoporphyrinogen IX oxidase (PPO) gene and/or a mutated sunflower protoporphyrinogen IX oxidase as defined above in connection with the plant of the present invention, and
c) electing or identifying a plant or seed comprising said gene and/or protoporphyrinogen IX oxidase.

The present invention further relates to a method for determining the germination rate of seeds, comprising
a) germinating a plurality of the seeds of the present invention in the presence of an effective amount of one or more PPO herbicides, and
b) determining the number of seeds that have germinated and the number of seeds that have not germinated, thereby determining the germination rate of the seeds.

### DETAILED DESCRIPTION OF THE PRESENT INVENTION

The inventors have screened sunflower plants grown from approximately more than 500.000 EMS mutagenized seeds for tolerance to PPO herbicide (Example 2). Out of the 500.000 screened plants, only one plant showed an acceptable tolerance to the PPO herbicide saflufenacil (Trade name: Kixor©). Specifically, it was found that a substitution of the phenylalanine residue at position 383 of the sunflower protoporphyrinogen IX oxidase 2 (PPO2) with an isoleucine residue (F383I) confers increased tolerance to saflufenacil to the sunflower plant (Examples 2 and 3). Further, it was shown that the mutation confers tolerance to other PPO herbicides as well (see e.g. Examples 7 and 8).

Seeds of the non-transgenic sunflower plant comprising a mutated protoporphyrinogen IX oxidase as described herein (Helianthus annuus L., HA452 inbred line, designated “21LHHA000892”) have been deposited on Apr. 22, 2022 at the National Collections of Industrial, Food and Marine Bacteria (NCIMB), Aberdeen, United Kingdom under the provisions of the Budapest treaty on the International Recognition of the Deposit of Microorganisms for the Purposes of Patent Procedure. The deposited seeds were assigned the accession number NCIMB 43974. The deposition of seeds was made only for convenience of the person skilled in the art and does not constitute or imply any confession, admission, declaration or assertion that deposited seed are required to fully describe the invention, to fully enable the invention or for carrying out the invention or any part or aspect thereof. Also, the deposition of seeds does not constitute or imply any recommendation to limit the application of any method of the present invention to the application of such seed or any material comprised in such seed, e.g. nucleic acids, proteins or any fragment of such nucleic acid or protein.

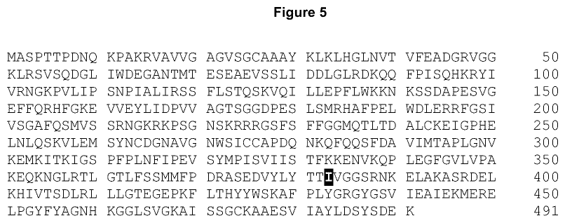

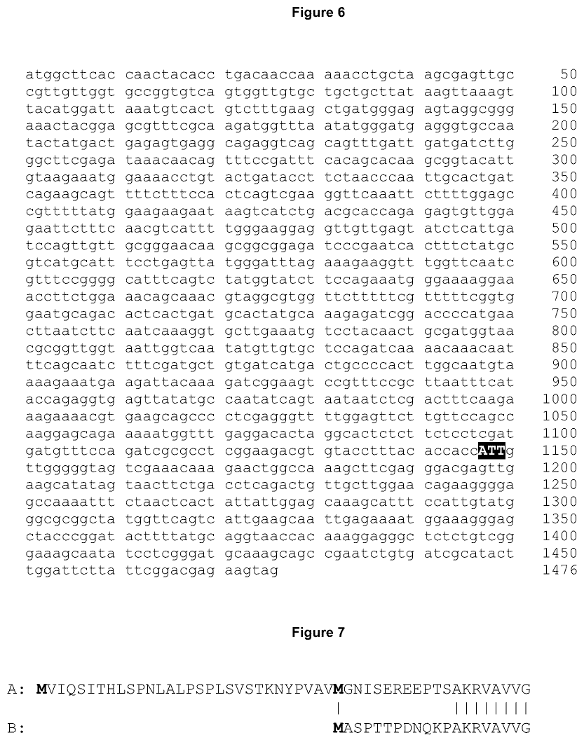

The protein sequence of the mutated sunflower protein is also shown in FIG. 5. The substitution at position 383 is highlighted. The substitution is the consequence of T to A transition in the codon for residue 383 of the wild-type PPO2 (TTT(F) to ATT(I) change, see also FIG. 6). In vitro studies with purified mutated protoporphyrinogen IX oxidase 2 showed that enzyme also confers tolerance to PPO herbicides other than saflufenacil.

The present invention, therefore, provides the first non-transgenic sunflower plant which shows tolerance to a variety of PPO inhibitors. The results of the studies of the present invention are surprising because the mutated sunflower protoporphyrinogen IX oxidase 2 is expressed under the control of the native PPO2 promoter which is a weak promoter. In contrast, the prior art teaches the use of strong constitutive promoters, such as the ubiquitin promoter. An in silico analysis showed that the sunflower PPO genes are much weaker expressed than the sunflower ubiquitin genes (see Example 9).

Further, the prior art teaches PPO mutations in the context of dual targeted proteins like PPO type II from Amaranthus which provide tolerant proteins to plastids and mitochondria, whereas PPO type II from sunflower is shown to lack the N-terminal extension shown to mediate the dual targeting for example in Amaranthus and spinach. Further, the results are surprising because sunflower plants are, in general, very susceptible to the PPO inhibitor saflufenacil. Therefore, it could not have been predicted that a PPO-herbicide tolerant sunflower plant could be obtained by EMS mutagenesis. Further, the whole plant tolerance could not have been predicted because of the multitude of factors at play at the whole plant level. Moreover, plants are subject to biotic (pests, weeds, etc.) and abiotic (weather, soil, etc.) stresses. These factors make it difficult to extrapolate from the biomolecule to the phenotypic level, particularly when it comes to herbicide tolerance.

Transgenic plants may not be suitable for all purposes. The inventors used a distinct technique for the generation of a PPO-herbicide tolerant plant.

Accordingly, the present invention relates to a non-transgenic sunflower plant comprising a mutated protoporphyrinogen IX oxidase (PPO) gene encoding a mutated sunflower protoporphyrinogen IX oxidase, wherein the mutated sunflower protoporphyrinogen IX oxidase comprises a substitution of phenylalanine (F) to isoleucine (I) at a position corresponding to residue 383 (F383I substitution) of the wild-type PPO2 polypeptide. Thus, the mutated PPO2 polypeptide shall comprise an isoleucine residue at a position corresponding to residue 383 (F383I substitution) of the wild-type PPO2 polypeptide. Preferably, the wild-type PPO2 polypeptide has an amino acid sequence as shown in SEQ ID NO: 4.

The articles “a” and “an” are used herein to refer to one or more than one (i.e., to at least one) of the grammatical object of the article. By way of example, “an element” means one or more elements.

As used herein, the word “comprising”, or variations such as “comprises” will be understood to imply the inclusion of a stated element, integer or step, or group of elements, integers or steps, but not the exclusion of any other element, integer or step, or group of elements, integers or steps.

Protoporphyrinogen IX oxidase (herein also referred to as “PPO” or “Protoporphyrinogen IX oxidase” catalyzes the seventh step in biosynthesis of protoporphyrin IX. In plants, protoporphyrin IX is the precursor to chlorophyll. Specifically, protoporphyrinogen IX oxidase (EC 1.3.3.4) catalyzes the dehydrogenation of protoporphyrinogen IX to form protoporphyrin IX. Preferably, the PPO polypeptide is a PPO2 polypeptide. For purposes herein, it is noted that PPO type II is used interchangeably with PPO2.

The term “mutated PPO gene” refers to a PPO nucleic acid molecule having a sequence that is mutated from a wild-type PPO gene, i.e. the wild-type PPO2 gene. The nucleic acid sequence of the sunflower wild-type PPO2 coding sequence is shown in SEQ ID NO: 3. The amino acid sequence of the wild-type PPO2 polypeptide is shown in SEQ ID NO: 4. As compared to the wild-type polypeptide, the mutated sunflower polypeptide shall comprise at least one mutation. Preferably, the mutated sunflower protoporphyrinogen IX oxidase comprises a substitution of phenylalanine (F) to isoleucine (I) at a position corresponding to residue 383 of SEQ ID NO: 4 or SEQ ID NO: 2 (F383I substitution). Thus, the mutated PPO oxidase shall comprise such a substitution at residue 383 relatively to SEQ ID NO: 4 (when aligned using blast).

Position 383 in the sunflower PPO2 polypeptide corresponds to position 420 in the Amaranthus tuberculatus type II PPO.

In an embodiment of the present invention, the mutated protoporphyrinogen IX oxidase comprises an amino acid sequence as shown in SEQ ID NO: 2. However, the present invention is not limited to SEQ ID NO: 2. Rather, the present invention pertains also to variants of the mutated protoporphyrinogen IX oxidase comprising an amino acid sequence as shown in SEQ ID NO: 2, provided that the variant comprises the substitution of phenylalanine (F) to isoleucine (I) at a position corresponding to residue 383 of SEQ ID NO: 2 or 4.

The expression “mutated amino acid” will be used below to designate the amino acid which is replaced by another amino acid, thereby designating the site of the mutation in the primary sequence of the protein.

The term “variant” with respect to a sequence (e.g., a polypeptide or nucleic acid sequence of the invention) is intended to mean substantially similar sequences. The variant polypeptide shall have protoporphyrinogen IX oxidase activity.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:
Seq A: AAGATACTG length: 9 bases
Seq B: GATCTGA length: 7 bases
   Hence, the shorter sequence is sequence B.

Producing a pairwise global alignment which is showing both sequences over their complete lengths results in

| | |
|---|---|
| Seq A: | AAGATACTG- |
| | \|\|\| \|\|\| |
| Seq B: | --GAT-CTGA |

The “|” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

| | |
|---|---|
| Seq A: | GATACTG- |
| | \|\|\| \|\|\| |
| Seq B: | GAT-CTGA |

Producing a pairwise alignment which is showing sequence A over its complete length according to the invention consequently results in:

| | |
|---|---|
| Seq A: | AAGATACTG |
| | \|\|\| \|\|\| |
| Seq B: | --GAT-CTG |

Producing a pairwise alignment which is showing sequence B over its complete length according to the invention consequently results in:

| | |
|---|---|
| Seq A: | GATACTG- |
| | \|\|\| \|\|\| |
| Seq B: | GAT-CTGA |

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the two aligned sequences over their complete length)*100. Thus, sequence identity in relation to comparison of two amino acid sequences according to this embodiment is calculated by dividing the number of identical residues by the length of the alignment region which is showing the two aligned sequences over their complete length. This value is multiplied with 100 to give “%-identity”. According to the example provided above, % identity is: (6/10)*100=60%.

Generally, amino acid sequence variants of the invention will have at least 70%, e.g., preferably at least 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, at least 98%, at least 99% or at least 99.5% polypeptide “sequence identity” to the polypeptide of SEQ ID NO: 2, provided that the encoded polypeptide comprises the substitution of phenylalanine (F) to isoleucine (I) at a position corresponding to residue 383 of SEQ ID NO: 2. Thus, the variant polypeptide shall comprise an isoleucine residue at the position corresponding to position 383 of SEQ ID NO: 2 (or SEQ ID NO: 4).

Similarly, nucleotide sequence variants of the invention will have at least 30, 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, at least 98%, at least 99% or at least 99.5% nucleotide “sequence identity” to the nucleotide sequence encoding a polypeptide of SEQ ID NO: 2, provided that the encoded polypeptide comprises the substitution of phenylalanine (F) to isoleucine (I) at a position corresponding to residue 383 of SEQ ID NO: 2 or 4.

Similarly, nucleotide sequence variants of the invention will have at least 30, 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, at least 98%, at least 99% or at least 99.5% nucleotide “sequence identity” to the nucleic acid sequence of SEQ ID NO: 1, provided that the encoded polypeptide comprises the substitution of phenylalanine (F) to isoleucine (I) at a position corresponding to residue 383 of SEQ ID NO: 2 or 4.

In a preferred embodiment, the mutated protoporphyrinogen IX oxidase comprises: an amino acid sequence as shown in SEQ ID NO: 2, or a is a variant thereof being at least 98%, such as at least 99% or at least 99.5% identical to SEQ ID NO: 2, with the proviso that the variant comprises a substitution of phenylalanine (F) to isoleucine (I) at a position corresponding to residue 383.

Further, it is envisaged that the mutated protoporphyrinogen IX oxidase (PPO) gene comprises
a) a nucleic acid sequence as shown in SEQ ID NO: 1, or
b) a nucleic acid sequence being at least 98%, such as at least 99% or at least 99.5% identical to SEQ ID NO: 1.

Moreover, it is envisaged that the mutated PPO polypeptide comprises not more than three, such as not more than two, such as not more than 1 mutations in addition to the F383I substitution.

SEQ ID NO: 1 and 3 are coding sequences, i.e. sequences which are translated. The sunflower PPO2 gene comprises many introns. It is to be understood that the sequences of these introns are not comprised by SEQ ID NO: 1 and 3, respectively. Thus, the expression that “the mutated protoporphyrinogen IX oxidase (PPO) gene comprises a nucleic acid sequence” shall mean that plant expresses a transcript comprising said sequence.

Gene stacking, also referred to as gene pyramiding, is the process of combining two or more genes of interest into a single plant. The combined traits resulting from this process are called stacked traits. When a stack is engineered or breed into a crop, the crop has better overall performance since a variety of genes for controlling different problems can in theory be stacked together. Moreover, gene stacking allows for better performance because if the resistance or tolerance conferred by a single gene breaks down, there is still a remaining gene that confers some benefit. Stacking can be achieved by transgenic approaches but also by using conventional breeding techniques.

In the context of weed management, genes conferring tolerance to commercial herbicides can be stacked to broaden the herbicidal mode of actions. For example, the glyphosate resistance gene has been stacked with genes conferring resistance to commercial herbicides.

Sunflower plants having the gene for a mutated PPO polypeptide hereof can also optionally be crossed to “stack” the PPO tolerance trait according to the current invention with other traits including, other herbicide tolerance traits. For example, the Clearfield (i.e. in Clearfield crops, the herbicide tolerance trait is conferred by a single point mutation in the acetohydroxyacid synthase (AHAS) gene (R gene), with an alanine to valine substitution at position 205 (Arabidopsis alignment) such that herbicides have reduced binding and inhibiting efficiency to the modified AHAS enzyme) and Clearfield Plus (i.e. the Clearfield Plus production system is a based on a single gene with higher levels of tolerance to imidazolinones) traits in sunflowers provide sunflowers with greater crop tolerance regardless of environmental stresses, improved weed control, oil content and grain yield. Elite cultivated sunflower lines possessing these traits could be combined with the mutated PPO trait of the present invention using breeding techniques known in the art. Moreover, the trait of PPO inhibitor tolerance of the present invention can be stacked with any other trait conferring herbicide tolerance or any other trait that provides for agronomic enhancement.

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents, such as seed obtained by selfing or crossing, hybrid plants and plant parts derived there from are encompassed herein, unless otherwise indicated. The term “plant” also encompasses plant cells, suspension cultures, callus tissue, embryos, meristematic regions, gametophytes, sporophytes, pollen and microspores, again wherein each of the aforementioned comprises the mutated PPO gene of the present invention.

The term “sunflower” as used herein, shall refer to any plant belonging to the genus Helianthus. In an embodiment, the term refers to a plant of the species Helianthus annuus. L.

In an embodiment, the sunflower plant is the domesticated sunflower, Helianthus annuus ssp. macrocarpus), including its oilseed-type and confection-type varieties.

As set forth elsewhere herein, the sunflower plant may be a sunflower plant which is resistant to at least one AHAS (acetohydroxyacid synthase)-inhibiting herbicide, such as to an AHAS-inhibiting herbicide selected from the group consisting of imidazolinone herbicides, sulfonylurea herbicides, triazolopyrimidine herbicides, pyrimidinyloxybenzoate herbicides, and sulfonylamino-carbonyltriazolinone herbicides. Therefore, the sunflower mutant may also comprise a mutated AHAS gene which confers resistance to said herbicide. Such mutated genes are described e.g. in WO 2008/124431 A1 (incorporated herein by reference).

In some embodiments, the mutated PPO gene is present in homozygous form in the plant (or part thereof).

As used herein, the term “homozygous” means a genetic condition existing when two identical alleles reside at a specific locus, but are positioned individually on corresponding pairs of homologous chromosomes in the cell. In contrast, the term “heterozygous” means a genetic condition existing when two different alleles reside at a specific locus, but are positioned individually on corresponding pairs of homologous chromosomes in the cell.

As used herein, the term “non-transgenic” refers to a plant or plant cell that does not have DNA derived from another organism inserted into its genome. Thus, the non-transgenic plant shall not have been produced by recombinant means. For example, the mutated PPO shall not have been introduced by transformation, such as Agrobacterium-mediated transformation. However, a non-transgenic plant or cell may have been produced by introducing a targeted mutation in the PPO2 gene, e.g. by gene editing.

Since the plant of the present invention is non-transgenic, it will be understood that the mutated PPO2 gene shall be at the same position in the sunflower genome as the wildtype PPO2 gene. Thus, the mutated PPO2 gene may be operably linked to the native (i.e. wild-type) promoter of the protoporphyrinogen IX oxidase (PPO2) gene which is known in the art and comprises a sequence as shown in SEQ ID NO: 5.

In an embodiment of the present invention, the native promoter comprises a nucleic acid sequence as shown in SEQ ID NO: 5, or a sequence being at least 90%, 91%, 92%, 93%, 94%, 95%, 96%, 97%, at least 98%, at least 99% or at least 99.5% identical to SEQ ID NO: 5, or a fragment thereof, such as a fragment having a length of at least 200, 300 or 500 bp.

Typically, the non-transgenic plant has not been exclusively obtained by means of an essentially biological process.

The plant of the present invention shall be tolerant to PPO-inhibiting herbicides. In an embodiment of the present invention, the trait of tolerance to PPO-inhibiting herbicides is an endogenous non-transformed trait. Thus, the mutated PPO gene shall not have been introduced by transformation of a transgene. In an embodiment of the present invention, the trait of tolerance to PPO-inhibiting herbicides is an endogenous non-transfected trait. Thus, the PPO gene shall not have been mutated by gene editing.

In an embodiment of the present invention, the plant has been produced by Ethyl methanesulfonate mutagenesis. Thus, the mutation in the PPO2 gene as referred to herein has been introduced by EMS (ethyl methanesulfonate) mutagenesis. Ethyl methanesulfonate (EMS) is a mutagenic compound that produces random mutations in genetic material by nucleotide substitution; particularly through G:C to A:T transitions induced by guanine alkylation.

In an embodiment of the present invention, the plant has been produced by radiation induced mutagenesis. Thus, the mutation in the PPO2 gene as referred to herein has been introduced by radiation induced mutagenesis.

Gene editing techniques may not be currently feasible in sunflower, but where available such techniques could be used to produce the plants of the invention. In an embodiment of the present invention, the plant may be thus produced by genome editing. Thus, the mutation in the PPO2 gene as referred to herein may be introduced by genome editing. Genome editing, as used herein, refers to the targeted modification of genomic DNA using sequence-specific enzymes (such as endonuclease, nickases, base conversion enzymes) and/or donor nucleic acids (e.g. dsDNA, oligo's) to introduce desired changes in the DNA. Sequence-specific nucleases that can be programmed to recognize specific DNA sequences include meganucleases (MGNs), zinc-finger nucleases (ZFNs), TAL-effector nucleases (TALENs) and RNA-guided or DNA-guided nucleases such as Cas9, Cpf1, CasX, CasY, C2c1, C2c3, certain argonout systems (see e.g. Osakabe and Osakabe, Plant Cell Physiol. 2015 March; 56 (3): 389-400; Ma et al., Mol Plant. 2016 Jul. 6; 9 (7): 961-74; Bortesie et al., Plant Biotech J, 2016, 14; Murovec et al., Plant Biotechnol J. 2017 Apr. 1; Nakade et al., Bioengineered 8-3, 2017; Burstein et al., Nature 542, 37-241; Komor et al., Nature 533, 420-424, 2016; all incorporated herein by reference). Donor nucleic acids can be used as a template for repair of the DNA break induced by a sequence specific nuclease, but can also be used as such for gene targeting (without DNA break induction) to introduce a desired change into the genomic DNA.

By using the above technologies, plants comprising a wild-type sunflower PPO2 can be converted to plants comprising the mutated PPO2 gene as referred to herein, thereby increasing the tolerance to PPO-inhibiting herbicides.

Thus, the non-transgenic sunflower plant of the present invention shall be resistant or tolerant to one or more PPO-inhibiting herbicides.

Generally, the term “herbicide” as used herein to mean an active ingredient that kills, controls or otherwise adversely modifies the growth of plants. The preferred amount or concentration of the herbicide is an “effective amount” or “effective concentration.” By “effective amount” and “effective concentration” is intended an amount and concentration, respectively, that is sufficient to kill or inhibit the growth of a similar, wild-type, plant, plant tissue, plant cell, or host cell, but that said amount does not kill or inhibit as severely the growth of the herbicide-resistant plants, plant tissues, plant cells, and host cells of the present invention. Typically, the effective amount of a herbicide is an amount that is routinely used in agricultural production systems to kill weeds of interest. Such an amount is known to those of ordinary skill in the art. Herbicidal activity is exhibited by herbicides useful for the present invention when they are applied directly to the plant or to the locus of the plant at any stage of growth or before planting or emergence. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted as is known in the art to promote non-selective or selective herbicidal action. Generally, it is preferred to apply the herbicide post emergence to relatively immature undesirable vegetation to achieve the maximum control of weeds.

In an embodiment, the effective amount is and amount which is effective to inhibit the growth of a wild-type sunflower plant.

In an embodiment, the herbicide is saflufenacil, and the effective amount is 1 to 50 a.i. g/ha.

In another embodiment, the herbicide is saflufenacil, and the effective amount is 2 to 25 a.i. g/ha.

In another embodiment, the herbicide is saflufenacil, and the effective amount is 5 to 15 a.i. g/ha.

By a “herbicide-tolerant” or “herbicide-resistant” plant, it is intended that a plant that is tolerant or resistant to at least one herbicide at a level that would normally kill, or inhibit the growth of, a normal or wild-type plant. By “herbicide-tolerant mutated PPO protein” or “herbicide-resistant mutated PPO protein”, it is intended that such a PPO protein displays higher PPO activity, relative to the PPO activity of the wild-type, i.e. the unmutated PPO protein, when in the presence of at least one herbicide that is known to interfere with PPO activity and at a concentration or level of the herbicide that is known to inhibit the PPO activity of the wild-type PPO protein. Furthermore, the PPO activity of such a herbicide-tolerant or herbicide-resistant mutated PPO protein may be referred to herein as “herbicide-tolerant” or “herbicide-resistant” PPO activity. The terms are used interchangeably herein.

In an embodiment, the tolerance to a PPO-inhibiting herbicide of the non-transgenic sunflower plant of the present invention is greater than that of a corresponding wild-type sunflower plant (i.e. a plant which does not comprise the F383I substitution).

Generally, if the PPO-inhibiting herbicides which can be employed in the context of the present invention, are capable of forming geometrical isomers, for example E/Z isomers, it is possible to use both, the pure isomers and mixtures thereof, in the compositions useful for the present the invention. If the PPO-inhibiting herbicides A as described herein have one or more centers of chirality and, as a consequence, are present as enantiomers or diastereomers, it is possible to use both, the pure enantiomers and diastereomers and their mixtures, in the compositions according to the invention. If the PPO-inhibiting herbicides A as described herein have ionizable functional groups, they can also be employed in the form of their agriculturally acceptable salts. Suitable are, in general, the salts of those cations and the acid addition salts of those acids whose cations and anions, respectively, have no adverse effect on the activity of the active compounds. Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four hydrogen atoms are replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, methylammonium, isopropylammonium, dimethylammonium, diisopropylammonium, trimethylammonium, heptylammonium, dodecylammonium, tetradecylammonium, tetramethylammonium, tetraethylammonium, tetrabutylammonium, 2-hydroxyethylammonium (olamine salt), 2-(2-hydroxyeth-1-oxy) eth-1-ylammonium (diglycolamine salt), di(2-hydroxyeth-1-yl)ammonium (diolamine salt), tris(2-hydroxyethyl)ammonium (trolamine salt), tris(2-hydroxypropyl)ammonium, benzyltrimethylammonium, benzyltriethylammonium, N,N,N-trimethylethanolammonium (choline salt), furthermore phosphonium ions, sulfonium ions, preferably tri (C₁-C₄-alkyl) sulfonium, such as trimethylsulfonium, and sulfoxonium ions, preferably tri (C₁-C₄-alkyl) sulfoxonium, and finally the salts of polybasic amines such as N, N-bis-(3-aminopropyl)methylamine and diethylenetriamine. Anions of useful acid addition salts are primarily chloride, bromide, fluoride, iodide, hydrogensulfate, methylsulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate and butyrate.

The PPO-inhibiting herbicides as described herein having a carboxyl group can be employed in the form of the acid, in the form of an agriculturally suitable salt as mentioned above or else in the form of an agriculturally acceptable derivative, for example as amides, such as mono- and di-C₁-C₆-alkylamides or arylamides, as esters, for example as allyl esters, propargyl esters, C₁-C₁₀-alkyl esters, alkoxyalkyl esters, tefuryl ((tetrahydrofuran-2-yl)methyl) esters and also as thioesters, for example as C₁-C₁₀-alkylthio esters. Preferred mono- and di-C₁-C₆-alkylamides are the methyl and the dimethylamides. Preferred arylamides are, for example, the anilides and the 2-chloroanilides. Preferred alkyl esters are, for example, the methyl, ethyl, propyl, isopropyl, butyl, isobutyl, pentyl, mexyl (1-methylhexyl), meptyl (1-methylheptyl), heptyl, octyl or isooctyl (2-ethylhexyl) esters. Preferred C₁-C₄-alkoxy-C₁-C₄-alkyl esters are the straight-chain or branched C₁-C₄-alkoxy ethyl esters, for example the 2-methoxyethyl, 2-ethoxyethyl, 2-butoxyethyl (butotyl), 2-butoxypropyl or 3-butoxypropyl ester. An example of a straight-chain or branched C₁-C₁₀-alkylthio ester is the ethylthioester.

Examples of PPO inhibiting herbicides which can be used according to the present invention are acifluorfen, acifluorfen-sodium, aclonifen, azafenidin, bencarbazone, benzfendizone, bifenox, butafenacil, carfentrazone, carfentrazone-ethyl, chlomethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, chlornitrofen, flumipropyn, fluoronitrofen, flupropacil, furyloxyfen, nitrofluorfen, ethyl [3-[2-chloro-4-fluoro-5-(1-methyl-6-trifluoromethyl-2,4-dioxo-1,2,3,4-tetrahydropyrimidin-3-yl) phenoxy]-2-pyridyloxylacetate (CAS 353292-31-6; S-3100), N-ethyl-3-2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100 03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4), 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0), 1-M ethyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione, methyl (E)-4-[2-chloro-5-[4-chloro-5-(difluoromethoxy)-1H-methyl-pyrazol-3-yl]-4-fluorophenoxy]-3-methoxy-but-2-enoate [CAS 948893-00-3], and 3-[7-Chloro-5-fluoro-2-(trifluoromethyl)-1H-benzimidazol-4-yl]-1-methyl-6-(trifluoromethyl)-1H-pyrimidine-2,4-dione (CAS 212754-02-4), and uracils of formula III

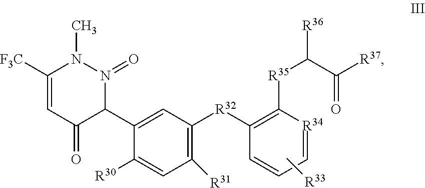

wherein
R³⁰ and R³¹ independently of one another are F, Cl, Br or CN; for example R³⁰ and R³¹ independently of one another are F, Cl or CN
R³² is O or S;
R³³ is H, F, Cl, CH₃ or OCH₃;
R³⁴ is CH or N;
R³⁵ is O or S;
R³⁶ is H, CN, CH₃, CF₃, OCH₃, OC₂H₅, SCH₃, SC₂H₅, (CO) OC₂H₅ or CH₂R³⁸, wherein R³⁸ is F, Cl, OCH₃, SCH₃, SC₂H₅, CH₂F, CH₂Br or CH₂OH; and
R³⁷ is (C₁-C₆-alkyl)amino, (C₁-C₆-dialkyl)amino, (NH) OR³⁹, OH, OR⁴⁰ or SR⁴⁰ wherein R³⁹ is CH₃, C₂H₅ or phenyl; and
R⁴⁰ is independently of one another C₁-C₆-alkyl, C₂-C₆-alkenyl, C₃-C₆-alkynyl, C₁-C₆-haloalkyl, C₁-C₆-alkoxy-C₁-C₆-alkyl, C₁-C₆-alkoxy-C₁-C₆-alkoxy-C₁-C₆-alkyl, C₂-C₆-cyanoalkyl, C₁-C₄-alkoxy-carbonyl-C₁-C₄-alkyl, C₁-C₄-alkyl-carbonyl-amino, C₁-C₆-alkylsulfinyl-C₁-C₆-alkyl, C₁-C₆-alkyl-sulfonyl-C₁-C₆-alkyl, C₁-C₆-dialkoxy-C₁-C₆-alkyl, C₁-C₆-alkyl-carbonyloxy-C₁-C₆-alkyl, phenyl-carbonyl-C₁-C₆-alkyl, tri (C₁-C₃-alkyl)-silyl-C₁-C₆-alkyl, tri (C₁-C₃-alkyl) silyl-C₁-C₆-alkenyl, tri (C₁-C₃-alkyl)-silyl-C₁-C₆-alkynyl, tri (C₁-C₃-alkyl) silyl-C₁-C₆-alkoxy-C₁-C₆-alkyl, dimethylamino, tetrahydropyranyl, tetrahydrofuranyl-C₁-C₃-alkyl, phenyl-C₁-C₆-alkoxy-C₁-C₆-alkyl, phenyl-C₁-C₃-alkyl, pyridyl-C₁-C₃-alkyl, pyridyl, phenyl,
which pyridyls and phenyls independently of one another are substituted by one to five substituents selected from the group consisting of halogen, C₁-C₃-alkyl or C₁-C₂-haloalkyl;
C₃-C₆-cycloalkyl or C₃-C₆-cycloalkyl-C₁-C₄-alkyl,
which cycloalkyls independently of one another are unsubstituted or substituted by one to five substituents selected from the group consisting of halogen, C₁-C₃-alkyl and C₁-C₂-haloalkyl;
including their agriculturally acceptable alkali metal salts or ammonium salts.

Further PPO-inhibiting herbicides that can be used according to the present invention are: 2-[2-chloro-5-[3-chloro-5-(trifluoromethyl)-2-pyridinyl]-4-fluorophenoxy]-2-methoxy-acetic acid methyl ester (CAS 1970221-16-9), 2-[2-[[3-chloro-6-[3,6-dihydro-3-methyl-2,6-dioxo-4-(trifluoromethyl)-1 (2H)-pyrimidinyl]-5-fluoro-2-pyridinyl]oxy]phenoxy]-acetic acid methyl ester (CAS 2158274-96-3), 2-[2-[3-chloro-6-[3,6-dihydro-3-methyl-2,6-dioxo-4-(trifluoromethyl)-1 (2H)-pyrimidinyl]-5-fluoro-2-pyridinyl]oxy]phenoxy]acetic acid ethyl ester (CAS 2158274-50-9), methyl 2-[[3-[2-chloro-5-[4-(difluoromethyl)-3-methyl-5-oxo-1,2,4-triazol-1-yl]-4-fluoro-phenoxy]-2-pyridyl]oxy]acetate (CAS 2271389-22-9), ethyl 2-[[3-[2-chloro-5-[4-(difluoromethyl)-3-methyl-5-oxo-1,2,4-triazol-1-yl]-4-fluoro-phenoxy]-2-pyridyl]oxy]acetate (CAS 2230679-62-4), 2-[[3-[[3-chloro-6-[3,6-dihydro-3-methyl-2,6-dioxo-4-(trifluoromethyl)-1 (2H)-pyrimidinyl]-5-fluoro-2-pyridinyl]oxy]-2-pyridinyl]oxy]-acetic acid methyl ester (CAS 2158275-73-9), 2-[3-[[3-chloro-6-[3,6-dihydro-3-methyl-2,6-dioxo-4-(trifluoromethyl)-1 (2H)-pyrimidinyl]-5-fluoro-2-pyridinyl]oxy]-2-pyridinyl]oxy]acetic acid ethyl ester (CAS 2158274-56-5), 2-[2-[[3-chloro-6-[3,6-dihydro-3-methyl-2,6-dioxo-4-(trifluoromethyl)-1 (2H)-pyrimidinyl]-5-fluoro-2-pyridinyl]oxy]phenoxy]-N(methylsulfonyl)-acetamide (CAS 2158274-53-2), 2-[[3-[[3-chloro-6-[3,6-dihydro-3-methyl-2,6-dioxo-4-(trifluoromethyl)-1 (2H)-pyrimidinyl]-5-fluoro-2-pyridinyl]oxy]-2-pyridinyl]oxy]-N-(methylsulfonyl)-acetamide (CAS 2158276-22-1), methyl 2-[2-[2-bromo-4-fluoro-5-[3-methyl-2,6-dioxo-4-(trifluoromethyl)pyrimidin-1-yl]phenoxy]phenoxy]-2-methoxy-acetate (CAS 2703795-90-6), 3-[2-chloro-5-[3,6-dihydro-3-methyl-2,6-dioxo-4-(trifluoromethyl)-1(2H)-pyrimidinyl]-4-fluorophenyl]-4,5-dihydro-5-methyl-5-isoxazolecarboxylic acid ethyl ester (CAS 1949837-17-5), methyl (2R)-2-[(E)-([2-chloro-4-fluoro-5-[3-methyl-2,6-dioxo-4-(trifluoromethyl)-3,6-dihydropyrimidin-1 (2H)-yl]phenyl]methylidene)amino]oxy]propanoate (CAS 2759011-88-4).

Preferred PPO-inhibiting herbicides that can be used according to the present invention are: Acifluorfen, acifluorfen-sodium, azafenidin, bencarbazone, benzfendizone, butafenacil, carfentrazone-ethyl, cinidon-ethyl, flufenpyr-ethyl, flumiclorac-pentyl, flumioxazin, fluoroglycofen-ethyl, fluthiacet-methyl, fomesafen, lactofen, oxadiargyl, oxadiazon, oxyfluorfen, pentoxazone, pyraflufen-ethyl, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl) phenoxy]-2-pyridyloxylacetate (CAS 353292-31-6; S-3100), N-ethyl-3-(2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4, trifludimoxazin), 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0); 1-M ethyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0), 3-[7-Chloro-5-fluoro-2-(trifluoromethyl)-1H-benzimidazol-4-yl]-1-methyl-6-(trifluoromethyl)-1H-pyrimidine-2,4-dione uracils of formula III.1 (corresponding to uracils of formula III, wherein R³⁰ is F, R³¹ is Cl, R³² is O; R³³ is H; R³⁴ is CH; R³⁵ is O and R³⁷ is OR⁴⁰)

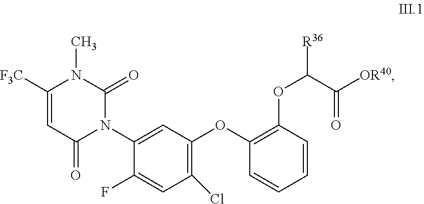

wherein
R³⁶ is OCH₃, OC₂H₅, SCH₃ or SC₂H₅; and
R⁴⁰ is C₁-C₆-alkyl, C₂-C₆-alkenyl, C₃-C₆-alkynyl, C₁-C₆-haloalkyl, C₁-C₆-alkoxy-C₁-C₆-alkyl, C₁-C₆-alkoxy-C₁-C₆-alkoxy-C₁-C₆-alkyl, C₁-C₃-cyanoalkyl, phenyl-C₁-C₃-alkyl, pyridyl-C₁-C₃-alkyl, C₃-C₆-cycloalkyl or C₃-C₆-cycloalkyl-C₁-C₄-alkyl,
which cycloalkyls are unsubstituted or substituted by one to five substituents selected from the group consisting of halogen, C₁-C₃-alkyl and C₁-C₂-haloalkyl; and
uracils of formula III.2 (corresponding to uracils of formula III, wherein R³⁰ is F; R³¹ is Cl; R³² is O; R³³ is H; R³⁴ is N; R³⁵ is O and R³⁷ is OR⁴⁰ with R⁴⁰ is C₁-C₆-alkyl)

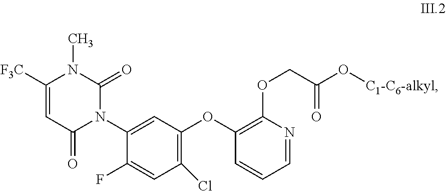

Particularly preferred PPO-inhibiting herbicides that can be used according to the present invention are: acifluorfen, acifluorfen-sodium, butafenacil, carfentrazone-ethyl, cinidon-ethyl, flumioxazin, fluthiacet-methyl, fomesafen, lactofen, oxadiargyl, oxyfluorfen, saflufenacil, sulfentrazone, ethyl [3-[2-chloro-4-fluoro-5-(1-methyl-6-trifluoromethyl-2,4-dioxo-1,2,3,4-tetrahydropyrimidin-3-yl)-phenoxy]-2-pyridyloxy]acetate (CAS 353292-31-6; S-3100), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (CAS 1258836-72-4, trifludimoxazin), and 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0), 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0), uracils of formula III.1.1 (corresponding to uracils of formula III, wherein R³⁰ is F, R³¹ is Cl, R³² is O; R³³ is H; R³⁴ is CH; R³⁵ is O, R³⁶ is OCH₃ and R³⁷ is OR⁴⁰)

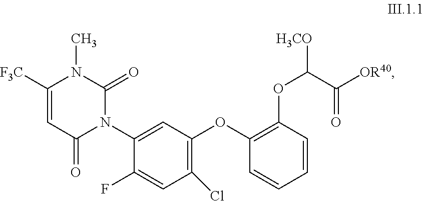

wherein
R⁴⁰ is C₁-C₆-alkyl, C₂-C₆-alkenyl, C₃-C₆-alkynyl, C₁-C₆-haloalkyl, C₁-C₆-alkoxy-C₁-C₆-alkyl, C₁-C₆-alkoxy-C₁-C₆-alkoxy-C₁-C₆-alkyl, C₁-C₃-cyanoalkyl, phenyl-C₁-C₃-alkyl, pyridyl-C₁-C₃-alkyl, C₃-C₆-cycloalkyl or C₃-C₆-cycloalkyl-C₁-C₄-alkyl,
which cycloalkyls are unsubstituted or substituted by one to five substituents selected from the group consisting of halogen, C₁-C₃-alkyl and C₁-C₂-haloalkyl;
is preferably CH₃, CH₂CH₂OC₂H₅, CH₂CHF₂, cyclohexyl, (1-methylcyclopropyl)methyl or CH₂ (pyridine-4-yl);
uracils of formula III.2.1 (corresponding to uracils of formula III, wherein R³⁰ is F; R³¹ is Cl; R³² is O; R³³ is H; R³⁴ is N; R³⁵ is O and R³⁷ is OR⁴⁰ with R⁴⁰ is CH₃)

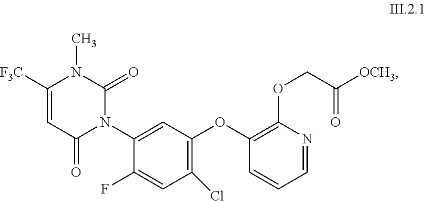 [SMILES: COC(=O)COc1ncccc1Oc1cc(-n2c(=O)cc(C(F)(F)F)n(C)c2=O)c(F)cc1Cl]

and
uracils of formula III.2.2 (corresponding to uracils of formula III, wherein R³⁰ is F; R³¹ is Cl; R³² is O; R³³ is H; R³⁴ is N; R³⁵ is O and R³⁷ is OR⁴⁰ with R⁴⁰ is C₂H₅)

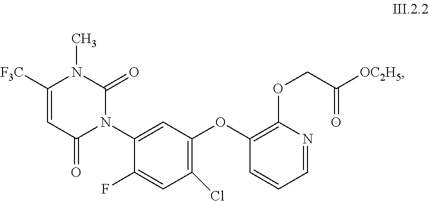 [SMILES: CCOC(=O)COc1ncccc1Oc1cc(-n2c(=O)cc(C(F)(F)F)n(C)c2=O)c(F)cc1Cl]

Especially preferred PPO-inhibiting herbicides are the PPO-inhibiting herbicides. 1 to A.14 listed below in table A

TABLE A

| | |
|---|---|
| A.1 | Acifluorfen |
| A.2 | Butafenacil |
| A.3 | carfentrazone-ethyl |
| A.4 | cinidon-ethyl |
| A.5 | Flumioxazin |
| A.6 | fluthiacet-methyl |
| A.7 | Fomesafen |
| A.8 | Lactofen |
| A.9 | Oxadiargyl |
| A.10 | Oxyfluorfen |
| A.11 | Saflufenacil |
| A.12 | Sulfentrazone |
| A.13 | ethyl [3-[2-chloro-4-fluoro-5-(1-methyl-6-trifluoromethyl-2,4- |
| | dioxo-1,2,3,4-tetra-hydropyrimidin-3-yl)phenoxy]-2- |
| | pyridyloxylacetate (CAS 353292-31-6) |
| A.14 | 1,5-dimethyl-6-thioxo-3-(2,2,7-trifluoro-3-oxo-4-(prop-2-ynyl)-3,4- |
| | dihydro-2H-benzo[b][1,4]oxazin-6-yl)-1,3,5-triazinane-2,4-dione |
| | CAS 1258836-72-4)-trifludimoxazin |

In an embodiment, the PPO-herbicide is carfentrazone-ethyl.

In another embodiment, the PPO-herbicide is flumioxazin.

In another embodiment, the PPO-herbicide is saflufenacil.

In another embodiment, the PPO-herbicide is trifludimoxazin.

In another embodiment, the PPO-herbicide is tiafenacil.

In another embodiment, the PPO-herbicide is methyl 2-[2-[2-bromo-4-fluoro-5-[3-methyl-2,6-dioxo-4-(trifluoromethyl)pyrimidin-1-yl]phenoxy]phenoxy]-2-methoxy-acetate (see e.g. Example 7).

In another embodiment, the PPO-herbicide is a composition comprising saflufenacil and trifludimoxazin. Compositions comprising Saflufenacil and trifludimoxazin were tested in the Examples section (see e.g. Examples 7 and 8).

The non-transgenic plant of the present invention shall comprise a phenotype of tolerance to one or more PPO-inhibiting herbicides, such as to saflufenacil. For example, the plant may comprise phenotype of tolerance to saflufenacil that is greater than 80% tolerance (i.e. less than 20% phytotoxicity) to 5 g a.i./ha saflufenacil, for example if applied at a V2-V8 stage or if applied at the 2-to-4 leaf-stage (i.e. at stage 12-14 of the BBCH (Biologische Bundesanstalt, Bundessortenamt und CHemische Industrie) scale—preferably, when grown on the field (see below). In some embodiments, the applied composition comprises 1% v/v MSO—methylated seed oil (in addition to saflufenacil).

In some embodiment, the plant may comprise phenotype of tolerance to 2 g a.i./ha saflufenacil, if applied at the 2-to-4 leaf-stage on the field.

Herbicide phytotoxicity can be assessed using a scale from 0% phytotoxicity (full tolerance) to 100% phytotoxicity (full susceptibility or absence of tolerance). Herbicide phytotoxicity is usually assessed in a population of (mutant) plants upon contacting the plant with the herbicide, e.g. a defined amount of herbicide, such as with saflufenacil at the 2-to-4 leaf-stage. Phytotoxicity can be assessed as described in the Examples section (see e.g. Example 4). In some embodiments, phytotoxicity is visually recorded for a population of plants after herbicide application on a per plot basis based on the rating scale table, such as a rating scale table shown in Table 2. The determined herbicide phytotoxicity can be used for calculating the herbicide tolerance. For example, a herbicide tolerance index can be calculated as described in Example 4, where 0% means no tolerance and 100% means full tolerance. A phenotype of tolerance that is greater than 80% tolerance means that the herbicide phytotoxicity is less than 20%. Thus, the plant (i.e. the population of plants) shows less than 20% phytotoxcity.

Interestingly, the mutated plants of the present invention survive and produce seeds after the application of an amount of 50 g a.i./ha of saflufenacil in the field. Thus, the plant of the present invention preferably survives and produces seeds under 50 g a.i./ha of saflufenacil on the field.

Furthermore, the present invention provides methods that involve the use of at least one PPO-inhibiting herbicide.

The present invention thus relates to a method for weed control at a plant cultivation site, comprising the steps of
a) providing a non-transgenic sunflower plant of the present invention at said plant cultivation site, and
b) applying an effective amount of one or more PPO-inhibiting herbicides at said site.

The term “weed control” is to be understood as meaning the killing of weeds and/or otherwise retarding or inhibiting the normal growth of the weeds. Weeds, in the broadest sense, are understood as meaning all those plants which grow in locations where they are undesired, e.g. (crop) plant cultivation sites. The weeds of the present invention include, for example, dicotyledonous and monocotyledonous weeds. Dicotyledonous weeds include, but are not limited to, weeds of the genera: Helianthus, Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera.

The cultivation site may be any site at which the sunflower is grown. In an embodiment, it is a greenhouse. In an alternative embodiment, it is a field. Preferably, the plant grown at the cultivation site, including the plant of the present invention and weed plants, are contacted with an effective amount of one or more PPO-inhibiting herbicides, e.g. by spraying.

The present invention also relates to a method for treating a plant, comprising the steps of
a) providing a non-transgenic sunflower plant of the present invention, and
b) applying an effective amount of one or more PPO-inhibiting herbicides to said plant.

In the above methods for weed control and for treating a plant, the at least one PPO-inhibiting herbicide can be applied by any method known in the art including, but not limited to, soil treatment, and foliar treatment. Prior to application, the PPO-inhibiting herbicide can be converted into the customary formulations, for example solutions, emulsions, suspensions, dusts, powders, pastes and granules. The use form depends on the particular intended purpose; in each case, it should ensure a fine and even distribution of the PPO-inhibiting herbicide.

By providing plants having increased tolerance to PPO-inhibiting herbicide, a wide variety of formulations can be employed for protecting plants from weeds, so as to enhance plant growth and reduce competition for nutrients. A PPO-inhibiting herbicide can be used by itself for pre-emergence, post-emergence, pre-planting, and at-planting control of weeds in areas surrounding the crop plants described herein, or a PPO-inhibiting herbicide formulation can be used that contains other additives. The PPO-inhibiting herbicide can also be used as a seed treatment. Additives found in a PPO-inhibiting herbicide formulation include other herbicides, detergents, adjuvants, spreading agents, sticking agents, stabilizing agents, or the like. The PPO-inhibiting herbicide formulation can be a wet or dry preparation and can include, but is not limited to, flowable powders, emulsifiable concentrates, and liquid concentrates. The PPO-inhibiting herbicide and herbicide formulations can be applied in accordance with conventional methods, for example, by spraying, irrigation, dusting, or the like.

In an embodiment, the at least one PPO-inhibiting herbicide is applied by spraying

Further, it is envisaged to apply the at least one PPO-inhibiting herbicide post-emergence. For example, the PPO-inhibiting herbicide can be applied about 10 to 14 days after emergence of the sunflower. Moreover, it is envisaged that the PPO-inhibiting herbicide is applied more than once.

Moreover, it is envisaged to apply the at least one PPO-inhibiting at 2-to-4 leaf-stage of the growth of the sunflower plant. This stage corresponds to stages 12-14 of the BBCH (Biologische Bundesanstalt, Bundessortenamt und CHemische Industrie) scale. The BBCH-scale is used to identify the phenological development stages of plants. The scale is e.g. described by Meier, U. (2001). “Growth stages of mono- and dicotyledonous plants”. BBCH Monograph. doi:10.5073/bbch0515, incorporated by reference herein. Further, the scale is described in LANCASHIRE et al. (Annals of Applied Biology. Volume 119, Issue3. Available in: https://doi.org/10.1111/j.1744-7348.1991.tb04895.x).

In accordance with the methods of the present invention, the methods comprise the application of an effective amount of one or more PPO-inhibiting herbicides at the cultivation site, or to said plant. Typically, the effective amount one or more PPO-inhibiting herbicides is and amount which is capable of controlling a weed of the genus Helianthus, Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and/or Apera.

The definitions and explanations given herein above preferably apply mutatis mutandis to the following.

The present invention further relates to a seed from the non-transgenic sunflower plant of the present invention, wherein said seed comprises the mutated protoporphyrinogen IX oxidase (PPO) gene. The seed shall be non-transgenic as well. In an embodiment, the mutated PPO gene is present in homozygous form in the seed. Furthermore, it is envisaged that the seed has on its surface an effective amount of one or more PPO-inhibiting herbicides.

The present invention further relates to a method for producing a product from sunflower seeds, said method comprising
a) growing the non-transgenic sunflower plant of the present invention at a plant cultivation site,
b) harvesting seeds from said plant, and
c) producing a product from the seeds harvested in step b.

Step a) of the above method may comprise the step of applying an effective amount of one or more PPO-inhibiting herbicides to said cultivation site as described elsewhere herein.

Preferably, the plant grown at the cultivation site, including the plant of the present invention and weed plants, are contacted with an effective amount of one or more PPO-inhibiting herbicides, e.g. by spraying.

Alternatively, the method may comprise the steps of:
a) providing a seed of the present invention, and
b) producing a product from the seed provided in step a).

In an embodiment, the product is bird feed.

In another embodiment, the product is seed meal.

In another embodiment, the product is sunflower oil. Accordingly, the above methods may comprise the extraction of sunflower oil from the harvested or provided seeds.

Thus, the present invention relates to a method for producing sunflower oil, comprising
a) growing the non-transgenic sunflower plant of the present invention at a plant cultivation site,
b) harvesting seeds from said plant, and
c) extracting sunflower oil from the seeds harvested in step b.

The present invention further relates to a method for producing sunflower oil, comprising
a) providing a seed of the present invention, and
b) extracting sunflower oil from the seed.

Further encompassed by the present invention is method for identifying a sunflower plant having improved resistance to one or more PPO-inhibiting herbicides, comprising
a) providing the seed of the present invention or a cell from the non-transgenic sunflower plant of the present invention,
b) subjecting said seed or cell to mutagenesis or transgenesis,
c) growing a plant from said seed or regenerating a plant from said cell, and
d) contacting the plant or a progeny thereof with an effective amount of one or more PPO-inhibiting herbicides, and
e) identifying a plant having improved resistance to one or more PPO-inhibiting herbicides.

The seeds of the present invention may be subjected to further mutagenesis. Therefore, plants may be identified which have an improved, i.e. increased, tolerance to PPO-inhibiting herbicides (such as to saflufenacil). The expressions “improved resistance” or “improved tolerance” mean that the tolerance to PPO-inhibiting herbicides (such as to saflufenacil) shall be increased as compared to the tolerance to PPO-inhibiting herbicides (such as to saflufenacil) of the non-transgenic sunflower plant of the present invention.

In an embodiment of the above method, the mutagenesis step is a step of random mutagenesis that utilizes tissue culture, a chemical mutagen, such as EMS, ionizing radiation, or fast neutron bombardment. For example, the seed or the cell may be subjected to EMS mutagenesis.

In an embodiment of the above method, the mutagenesis step is based on genome editing.

Since the plants to be identified by the above method shall have an improved tolerance to PPO herbicides, the plant or the progeny thereof are preferably contacted in step d) with an amount of a PPO herbicide which is higher than the amount tolerated by the plant of the present invention. For example, the effective amount of saflufenacil in the context of the method for identifying a sunflower plant having improved resistance may be an amount of 25 to 50 a.i. kg/ha.

The present invention further relates to a method for identifying and/or selecting a sunflower plant or seed having resistance to one or more PPO-inhibiting herbicides, comprising
a) providing a biological sample from a non-transgenic sunflower plant or seed of the present invention,
b) identifying or detecting in said sample the presence of a mutated protoporphyrinogen IX oxidase (PPO) gene and/or a mutated sunflower protoporphyrinogen IX oxidase as defined above in connection with the plant of the present invention, and
c) selecting or identifying a plant or seed comprising said gene and/or oxidase.

The present invention further relates to a method for determining the germination rate of seeds, comprising
a) germinating a plurality of the seeds, such as at least 100 seeds, of the present invention in the presence of an effective amount of one or more PPO herbicides, and
b) determining the number of seeds that have germinated and the number of seeds that have not germinated, thereby determining the germination rate of the seeds. The present invention also relates to a method of producing a sunflower plant that is resistant to one or more PPO inhibitors, the method comprising:
a) crossing a first sunflower plant with a second sunflower plant, where the first sunflower plant comprises in its genome at least one copy of a first allele of a mutated PPO2 gene encoding a mutated sunflower protoporphyrinogen IX oxidase, wherein the mutated sunflower protoporphyrinogen IX oxidase comprises a substitution of phenylalanine (F) to isoleucine (I) at a position corresponding to residue 383 relative to SEQ ID NO: 2 (F383I substitution); and
b) selecting resulting plants from such crossing that are tolerant to a level of PPO-inhibiting herbicide which prevents or inhibits the growth of a wild-type sunflower plant. The PPO herbicide has been defined above. The definition applies accordingly. In an embodiment, the herbicide is saflufenacil.

Preferably, the herbicide tolerance of the first sunflower plant is not developed through transgenic means. For example the first sunflower plant is developed by mutagenesis, e.g. EMS mutagenesis.

Finally, present invention relates to sunflower plant tolerant to a level of PPO herbicide that prevents or inhibits the growth of a wild-type sunflower plant, the PPO herbicide tolerant sunflower plant developed by crossing a herbicide tolerant sunflower plant having a mutation at F383I with a wildtype plant.

In the following, preferred embodiments of the present invention are disclosed. The definitions and explanations provided above apply mutatis mutandis.
1. A non-transgenic sunflower plant comprising a mutated protoporphyrinogen IX oxidase (PPO) gene encoding a mutated sunflower protoporphyrinogen IX oxidase, wherein the mutated sunflower protoporphyrinogen IX oxidase comprises a substitution of phenylalanine (F) to isoleucine (I) at a position corresponding to residue 383 relative to SEQ ID NO: 2 (F383I substitution).
2. The non-transgenic sunflower plant of embodiment 1, wherein the non-transgenic plant has been obtained by means other than exclusively an essentially biological process.
3. The non-transgenic sunflower plant of embodiments 1 or 2, wherein the mutated protoporphyrinogen IX oxidase comprises: an amino acid sequence as shown in SEQ ID NO: 2, or a variant thereof being at least 98%, or at least 99% or at least 99.5% identical to SEQ ID NO: 2, with the proviso that the variant comprises a substitution of phenylalanine (F) to isoleucine (I) at a position corresponding to residue 383.
4. The non-transgenic sunflower plant of any one of embodiments 1 to 3, wherein the mutated protoporphyrinogen IX oxidase (PPO) gene comprises
   a) a nucleic acid sequence as shown in SEQ ID NO: 1, or
   b) a nucleic acid sequence being at least 98%, or at least 99% or at least 99.5% identical to SEQ ID NO: 1.
5. The non-transgenic sunflower plant of any one of embodiments 1 to 4, wherein said plant possesses a phenotype of tolerance one or more PPO-inhibiting herbicides which tolerance is greater than that of a corresponding wild-type sunflower plant.
6. The non-transgenic sunflower plant of any one of embodiments 1 to 5, wherein the mutated PPO gene is present in homozygous form.
7. The non-transgenic sunflower plant of any one of embodiments 1 to 6, wherein the PPO gene has been mutated by EMS (ethyl methanesulfonate) mutagenesis.
8. The non-transgenic sunflower plant of any one of embodiments 1 to 6, wherein the PPO gene has been mutated by radiation induced mutagenesis.
9. The non-transgenic sunflower plant of any one of embodiments 1 to 6, wherein the PPO gene has been mutated by genome editing.
10. The non-transgenic sunflower plant of any one of embodiments 1 to 9 comprising on its leaves an effective amount of one or more PPO-inhibiting herbicides.
11. The non-transgenic sunflower plant of any one of embodiments 5 to 10, wherein the one or more PPO-inhibiting herbicides are selected from the group consisting of: carfentrazone-ethyl, flumioxazin, saflufenacil and/or trifludimoxazin herbicides, and combinations thereof.
12. The non-transgenic sunflower plant of any one of embodiments 1 to 11, wherein the plant comprises a phenotype of tolerance to a level of saflufenacil that would prevent or inhibit the growth of a wild-type plant.
13. The non-transgenic sunflower plant of any one of embodiments 1 to 12, wherein the plant comprises a phenotype of tolerance to saflufenacil that is greater than 80% tolerance to 5 g a.i./ha saflufenacil if applied at a V2-V8 stage.
14. The non-transgenic sunflower plant of any one of embodiments 1 to 13, wherein the gene is operably linked to the native promoter of the protoporphyrinogen IX oxidase (PPO) gene.
15. A seed from the non-transgenic sunflower plant of any one of embodiments 1 to 14, wherein said seed comprises the mutated protoporphyrinogen IX oxidase (PPO) gene.
16. The seed of embodiment 15 having on its surface an effective amount of one or more PPO-inhibiting herbicides
17. A method for weed control at a plant cultivation site, comprising the steps of
   a) providing a non-transgenic sunflower plant according to any one of embodiments 1 to 14 at said plant cultivation site, and
   b) applying an effective amount of one or more PPO-inhibiting herbicides at said site.
18. The method of embodiment 17, wherein said effective amount is effective to inhibit the growth of a wild-type sunflower plant.
19. A method for treating a plant, comprising the steps of
   a) providing a non-transgenic sunflower plant according to any one of embodiments 1 to 14, and
   b) applying an effective amount of an agronomically acceptable composition to said plant.
20. The method of embodiment 19, wherein the agronomically acceptable composition comprises one or more PPO-inhibiting herbicides.
21. The method of any one of embodiments 17 to 20 wherein the effective amount is an amount which is capable of controlling a weed of the genus Helianthus, Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and/or Apera.
22. The method of any one of embodiments 17 to 21, wherein the one or more PPO-inhibiting herbicides comprises saflufenacil, and wherein the effective amount is 1 to 50 a.i. g/ha, such as 2 to 25, such as 5 to 15 a.i. g/ha.
23. A method for producing sunflower oil, comprising
   a) growing the non-transgenic sunflower plant according to any one of embodiments 1 to 14 at a plant cultivation site,
   b) harvesting seeds from the plant, and
   c) extracting sunflower oil from the seeds harvested in step b.
24. The method of embodiment 23, wherein the method comprises the application of an effective amount of one or more PPO-inhibiting herbicides, such as an amount as defined in embodiment 17, 19 or 21, at the cultivation site.
25. A method for producing sunflower oil, comprising
   a) providing a seed according to embodiment 15, and
   b) extracting sunflower oil from the seed.
26. A method for identifying a sunflower plant having improved resistance to one or more PPO-inhibiting herbicides, comprising
   a) providing the seed of embodiment 15 or a cell from the non-transgenic sunflower plant according to any one of embodiments 1 to 14,
   b) subjecting said seed or cell to mutagenesis or transgenesis,
   c) growing a plant from said seed or regenerating a plant from said cell, and
   d) contacted the plant or a progeny thereof with an effective amount of one or more PPO-inhibiting herbicides, and
   e) identifying a plant having improved resistance to one or more PPO-inhibiting herbicides.
27. The method of embodiment 24, wherein the mutagenesis step is a step of random mutagenesis that may be achieved via tissue culture, chemical or physical mutagenesis. The chemical and physical seed mutagenesis steps may employ the use of chemical and physical mutagens, such as EMS and ionizing radiation, respectively.
28. The method of embodiment 24, wherein the mutagenesis step is based on genome editing.
29. A method for identifying and/or selecting a sunflower plant or seed having resistance to one or more PPO-inhibiting herbicides, comprising
   a) providing a biological sample from a non-transgenic sunflower plant of any one of embodiments 1 to 14, or from a seed of any of embodiments 15-16,
   b) identifying or detecting in said sample the presence of a mutated protoporphyrinogen IX oxidase (PPO) gene and/or a mutated sunflower protoporphyrinogen IX oxidase as defined in the preceding embodiments, and
   c) selecting or identifying a plant or seed comprising said gene and/or oxidase.
30. A method for determining the germination rate of seeds, comprising
   a) germinating a plurality of seeds of embodiment 15 in the presence of an effective amount of one or more PPO herbicides, and
   b) determining the number of seeds that have germinated and the number of seeds that have not germinated, thereby determining the germination rate of the seeds.
31. A method of producing a sunflower plant that is resistant to one or more PPO inhibitors, the method comprising:
   a) crossing a first sunflower plant with a second sunflower plant, where the first sunflower plant comprises in its genome at least one copy of a first allele of a mutated PPO2 gene encoding a mutated sunflower protoporphyrinogen IX oxidase, wherein the mu-tated sunflower protoporphyrinogen IX oxidase comprises a substitution of phenylal-anine (F) to isoleucine (I) at a position corresponding to residue 383 relative to SEQ ID NO: 2 (F383I substitution); and
   b) selecting resulting plants from such crossing that are tolerant to a level of PPO-inhibiting herbicide which prevents or inhibits the growth of a wild-type sunflower plant.
32. The method of embodiment 29, wherein the herbicide is saflufenacil.
33. The method of embodiment 29 or 30, wherein the herbicide tolerance of the first sunflower plant is not developed through transgenic means.
34 The method of any one of embodiments 29 to 31, wherein the first sunflower plant has been developed by mutagenesis.
35. A sunflower plant tolerant to a level of PPO herbicide that prevents or inhibits the growth of a wild-type sunflower plant, the PPO herbicide tolerant sunflower plant developed by crossing a herbicide tolerant sunflower plant having a mutation at F383I with a wildtype plant.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

All patents, patent applications, and publications or public disclosures (including publications on internet) referred to or cited herein are incorporated by reference in their entirety.

### SEQUENCES

SEQ ID NO: 1: nucleic acid sequence of mutated sunflower PPO2 gene (with F383I mutation, coding sequence)
SEQ ID NO: 2: amino acid sequence of mutated sunflower PPO2 protein (with F383I mutation)
SEQ ID NO: 3: nucleic acid sequence of wild-type sunflower PPO2 gene (coding sequence)
SEQ ID NO: 4: amino acid sequence of wild-type sunflower PPO2 protein
SEQ ID NO: 5 native promoter of the sunflower PPO2 gene

The invention will be further described with reference to the examples described herein; however, it is to be understood that the invention is not limited to such examples.

### Example 1: Wild Type Sunflower is Extremely Sensitive to PPO Inhibiting Herbicides, Such as Saflufenacil (Kixor™)

Research was conducted to evaluate the sensitivity of sunflower to the herbicide comprising the active ingredient (AI) saflufenacil (Trade name: Kixor™). The herbicide was applied in 2 field trials at different rates ranging from 0.91 g to 14 g a.i./ha (with 0.5-1% MSO-methylated seed oil-adjuvant) to 2 commercial sunflower varieties [a) a conventional (non-herbicide tolerant) sunflower (“Edison”) which is a linoleic oil-type sunflower, and b) a herbicide tolerant sunflower (“ES Coloris Clearfield which is a linoleic oil-type sunflower] and a public inbred line HA452, from the USDA (United States Department of Agriculture) National Plant Germplasm System (NPGS), deposited under Accession No. PI 642771. The treated plants were at the 2-to-4 leaf-stage (BBCH12-14). Trials were set up in a randomized complete block design with 3 replications and rated for herbicide phytotoxicity (percentage damage) 3, 7, 10 and 14 days after treatment (see also Example 4 for more details). Herbicide phytotoxicity ranges from 0% phytotoxicity (full tolerance) to 100% phytotoxicity (full susceptibility or absence of tolerance).

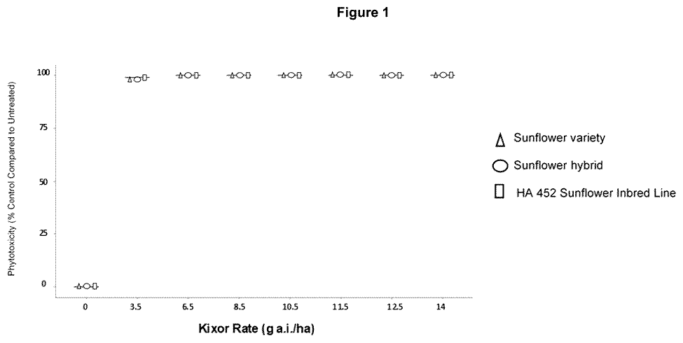

In the first field trial (trial 1), all three sunflower materials were completed controlled (i.e. killed) at the lowest saflufenacil rate after 14 days (FIG. 1). Full control of all sunflower materials was even observed at the first phytotoxicity evaluation time point, 3 days after treatment (DAT) with saflufenacil (results not shown).

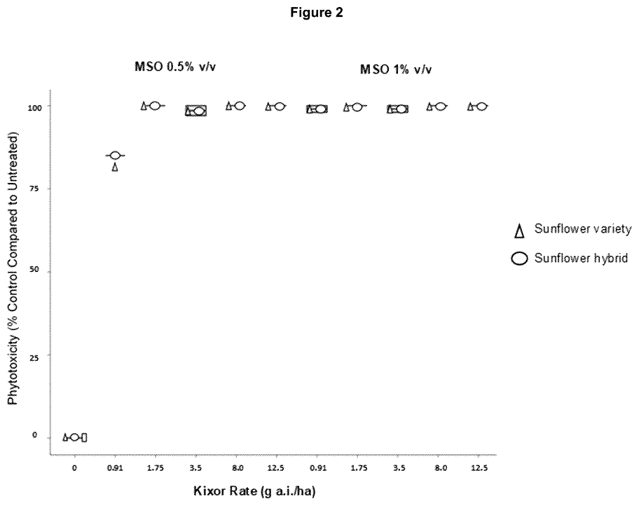

The second field trial (trial 2) compared the adjuvant MSO at two concentrations: 1% v/v, concentration used by the farmer and 0.5%, half the concentration (FIG. 2). With the MSO concentration of 0.5% v/v, which lowers the general herbicidal efficacy of saflufenacil in weeds, control of all sunflower materials was around 80% at the lowest rate of saflufenacil (0.91 g a.i./ha) and 100% at 1.75 g a.i./ha and higher (FIG. 2). With a concentration of 1% MSO v/v, again total control was observed at all applied rates.

The expected use field rate of saflufenacil (Kixor™) for an herbicide tolerant sunflower product is currently considered to be in the range of 25 g a.i./ha, 25 times larger than 0.91 g a.i./ha, which gave in the trials 100% phytotoxicity at 1% v/v MSO and a high control (80%), at half of the adjuvant concentration (MSO 0.5% v/v) was observed.

The field trials confirmed what has been already known: conventional sunflowers are very susceptible to the herbicide saflufenacil, even at very low rates. This leads to challenges to identify and confirm a mutation in the target PPO2 gene of sunflowers that confers high levels of herbicide tolerance to saflufenacil and other PPO inhibiting herbicides in general.

### Example 2: Field Forward Mutant Screening for Herbicide Tolerance

Seeds of the HA452 genotype were soaked with 0.08% N-ethyl-N-nitrosourea (ENU). This chemical mutagen is known to generate base pair changes A:T->T:A, A:T->G:C and G:C->A:T. Seed was harvested on the surviving treated plants, further increased and pooled to one seedlot.

A forward field screening for saflufenacil herbicide tolerant mutants was set up in Limburgerhof, Germany.

On Jun. 5, 2019, approximately 75 kg sunflower seeds (which we estimate to be more than a half million seeds) were planted. The field selection area comprised around 2 hectares. Plant emergence took place on Jun. 13, 2019. The target herbicide, saflufenacil (Kixor™), was sprayed in post-emergence of sunflower plants. Two saflufenacil applications were done in the field; the first application being at a rate of 12.5 g a.i./ha, and the second (one week later), being at a rate of 20 g a.i./ha. The first and second saflufenacil applications took place on Jun. 24, 2019 and Jul. 1, 2019, respectively.

Throughout the 21 days after the second herbicide treatment, the selection field was inspected for mutants that tolerated the two treatments of saflufenacil. Mutants were selected in 2 days, July 16th and 23rd. A total of 47 mutants were selected. The selected mutant plants were transplanted into pots in the same day they were selected, and the pots were kept in the greenhouse for seed production.

### Example 3

As it is not uncommon that in forward field mutant screening for herbicide tolerance ‘false’ tolerant plants are selected, the field candidates were reassessed in the greenhouse. Field harvested seeds of the mutant lead LIH16358 were planted in the greenhouse on Nov. 26, 2019, in Limburgerhof, Germany. The greenhouse trial had the objective to verify if the progeny of mutant lead LIH16358 would survive treatment with saflufenacil. Seeds of the susceptible wild type (HA452) line were planted in the same greenhouse trial as the susceptible reference. Saflufenacil was sprayed at 2.5 g a.i./ha on Dec. 10, 2019. The 2.5 g a.i./ha of saflufenacil corresponds to 12.5 g a.i./ha in field conditions. The final phytotoxicity evaluation in the treated plants was done on Dec. 24, 2019.

The first generation progeny of the mutant lead LIH16358, although showing some herbicide damage survived saflufenacil treatment, while the respective treated wild type reference plants were completely controlled and dead. Mutant survivors increasingly recovered after saflufenacil treatment and produced seeds.

The tolerant progeny individuals of mutant lead LIH16358 produced seeds, which were planted in a follow up greenhouse trial on May 19, 2020. The plants were sprayed with 2.0 g a.i./ha of saflufenacil on Jun. 3, 2020. Phytotoxicity was assessed for individual plants using the scoring system as shown in Table 2 in Example 4. Second generation progenies survived saflufenacil treatment, with individuals showing phytotoxicity as low as 25% at 14 Days after treatment (DAT). The respective wild type treated plants were completely controlled at 14 DAT, with phytotoxicity of 100% and were completely dead.

The nine plants with the lowest phytotoxicity percentages were leaf-sampled and had the whole PPO1 and PPO2 genes sequenced to assess whether single nucleotide polymorphism(s) was/were present in any of the PPO gene sequences, compared to the respective wild type reference PPO sequences. Results revealed that all the sequenced mutants had a single nucleotide polymorphism (SNP) in the PPO2 gene, namely a T to A transition in the codon for residue 383 of the wild-type PPO2 which resulted in a TTT(F) to ATT(I) change. The SNP caused a phenylalanine to isoleucine substitution in position 383 of the PPO2 protein, referred to as PPO2_F383I mutation.

TABLE 1
Sequencing Results List:

| Sample | | Well | sequencing results | |
|---|---|---|---|---|
| # | Plant ID | Position | PPO1 | PPO2 |
| 1 | 6-1 | A2 | wild type | F3831 homozygous |
| 2 | 6-5 | B2 | wild type | F3831 homozygous |
| 3 | 8-2 | C2 | wild type | F3831 heterozygous |
| 4 | 8-4 | D2 | wild type | F3831 heterozygous |
| 5 | 2-1 | E2 | wild type | F3831 homozygous |
| 6 | 1-1 | F2 | wild type | F3831 homozygous |
| 7 | 1-3 | G2 | wild type | F3831 homozygous |
| 8 | LIH17781-7 | H2 | wild type | F3831 homozygous |
| 9 | LIH17781-13 | A3 | wild type | F3831 homozygous |

### Example 4: Field Activities in 2021

M6 seed of eleven lines all originating from the nine plants carrying the PPO2_F383I mutation but with a different pedigree were assessed in an advanced field trial. The advanced field trial was manually planted in Utrera, Spain, on May 3, 2021. One row per mutant entry per plot was considered, and thirty-two seeds were planted per mutant entry per row per plot. Saflufenacil was applied in four increasing rates (0×: 0 g a.i/ha, 0.5×=12.5 g a.i./ha; 1×=25 g a.i./ha and 2×=50 g a.i./ha) when plants were at the BBCH12-14 growth stage. Saflufenacil application took place on Jul. 26, 2021. The field trial design was a strip-block design with four replications.

Phytotoxicity evaluations were done at 2, 5, 12, 19 and 26 days after treatment (DAT) with saflufenacil. As discussed previously, phytotoxicity of the mutant entries and wild type control were scored in each of the evaluation days after saflufenacil treatment. Eleven mutant lines were tested. The field tested one mutant line per row 32 seeds per row, for each saflufenacil rate treatment and replication, this means every plot was made of a single row, so the plot was the row for each mutant line. The phytotoxicity % score was given for each row in comparison to the untreated row of the same mutant line (control), meaning all 11 mutant lines were assessed for phytotoxicity % in all treatments and replications. Phytotoxicity was visually recorded at each evaluation time after saflufenacil application on a per plot basis based on the rating scale table (below).

TABLE 2
Phytotoxicity percentage score:

| Phytotoxicity | |
|---|---|
| (%) | Rating Scale |
| 0% | No plant injury. Treated and non-treated plants cannot be distinguished |
| 1-5% | Some plant injury, similar effect not seen in the non-treated plants |
| 6-10% | Plant injury slightly visible |
| 11-20% | Plant injury recognized as herbicide injury, fluctuation in height |
| 21-30% | Plant injury includes stunting, necrosis, chlorosis. Plant injury easily |
| | visible even from a distance |
| 31-40% | Plant injury easily recognized as herbicide injury even from a distance. |
| | Multiple symptoms include stunting, significant chlorosis, bleaching, |
| | chlorosis |
| 41-50% | Stunting, 40-50% height reduction, growing point stunted, moderate |
| | improper leaf unfolding, leaf necrosis. |
| 51-60% | Advanced chlorosis necrosis of growing point, improper leaf unfolding and |
| | stunting, malformed, puckered leaves and stems. Delayed, but continued |
| | emergence of growing points, loss of apical dominance |
| 61-70% | Advanced irregular development, tight clumping, irregular expansion of all |
| | leaves emerging subsequent to application |
| 81-90% | All growing points dead or severely chlorotic, necrotic, no growth, |
| | chlorosis, necrosis in oldest leaves, plant will, more than likely, die |
| 91-100% | Dead or greater than 90% of plant tissue necrotic |

To better reflect the tolerance of surviving plants, data were expressed in percent herbicide tolerance efficacy by using the formula below:

$$Herbicide Tolerance Efficacy ( \% ) = \frac{[ Phytotoxicity {( \% )}_{wild type} ] - [ Phytotoxicity {( \% )}_{mutant} ]}{[ Phytotoxicity {( \% )}_{wild type} ]} * 100$$

where:
0% means no tolerance
100% means full tolerance

The phytotoxicity difference between the wild type reference (susceptible) and the mutant progeny individuals after treatment reflects better how tolerant the mutant individuals are than the raw phytotoxicity scores. Saflufenacil phytotoxicity of the eleven PPO2_F383I mutant entries is higher than 15% at peak, compared to their untreated controls.

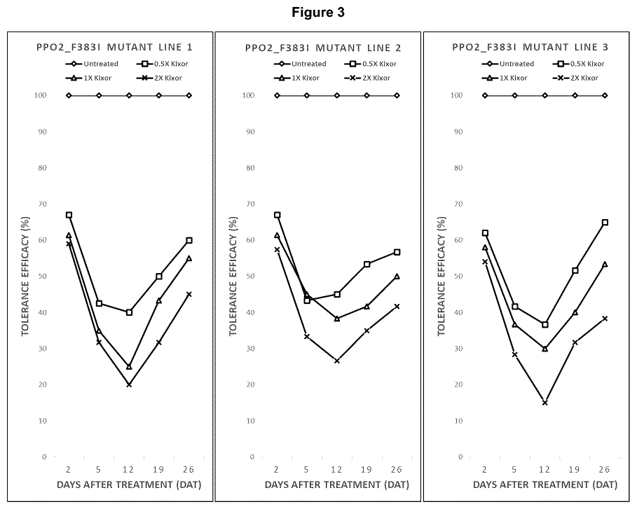

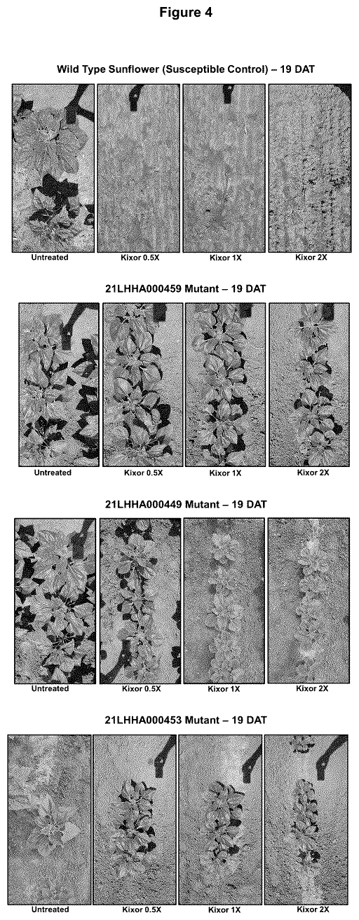

All eleven PPO2_F383I mutant lines showed a high tolerance efficacy (%) at all rates of the saflufenacil. Results of three lines are shown in FIGS. 3 and 4. At 12 DAT, tolerance efficacy decreases for all mutant entries, which start to significantly recover at 19 DAT for all rates, including the highest rate of 2×.

In addition, days to 50% flowering was also scored. Herbicide treated mutants reached 50% flowering z maximum of a few days before the respective untreated controls. For PPO2_F383I mutant line 1 the number of days to 50% flowering did not change among untreated and saflufenacil rates (data not shown). Tolerance of mutant entries is shown through plant survival, plant recovery and flowering, which was not impacted by more than 5 days across entries and saflufenacil rates.

Plant height was assessed when flowering was completed. The assessment was done at on Jul. 16, 2021, at 73 DAT. In general, untreated plants showed a greater plant height compared to treated plants. However, in PPO2_F383I mutant line 4, the difference between untreated and 0.5× and 1× was marginal.

### Example 5: In Vitro Assay

To test whether the PPO2_F383I is the main cause of the observed saflufenacil tolerance, we used an in vitro to compare the inhibition potency (IC50) of different PPO inhibiting herbicides between wild-type and mutant PPO2 enzyme. The in vitro assay assesses the oxidation of protoporphyrinogen IX to protoporphyrin IX, catalyzed by the PPO enzyme. Both the wild type and mutant PPO2 proteins were heterologously expressed in E. coli and purified. Purified protein was mixed with different concentrations of the herbicide ranging from 10⁻⁵M to 10⁻¹¹M and protein activity was measured. Protein activity is defined as the fluorescence per minute that the reaction generates. In the reaction, the purified PPO enzyme is mixed with substrate protoporphyrinogen IX and the herbicide. The product of the reaction is excited at 405 nm and produces a fluorescence at 630 nm which we detect. IC50 (in Mol) and tolerance factor were calculated.

TABLE 3
In vitro inhibition experiment

| | Wild type | PPO2_F383I | Tolerance |
|---|---|---|---|
| Herbicide | IC50 (mol) | IC50 (mol) | factor |
| Tiafenacil | 9.30E−10 | 1.30E−06 | 1398 |
| Saflufencacil (Kixor ™) | 1.46E−09 | 4.23E−06 | 2897 |
| Trifludimoxazin (Tirexor ™) | 1.40E−09 | 4.56E−06 | 3257 |
| Saflufenacil + | 7.97E−10 | 1.28E−07 | 161 |
| Trifludimoxazin | | | |

IC50 values for PPO2_F383I higher than 10⁻⁶M or a tolerance factor of around 1000 or higher are considered as highly tolerant. Results confirm that the PPO2_F383I mutation is the cause of the saflufenacil tolerance: IC50 is 4,36×10⁻⁶M and tolerance factor is 2897. The F383I mutants also show tolerance to other herbicides.

### Example 6: In Silico Analysis of Sunflower PPO2

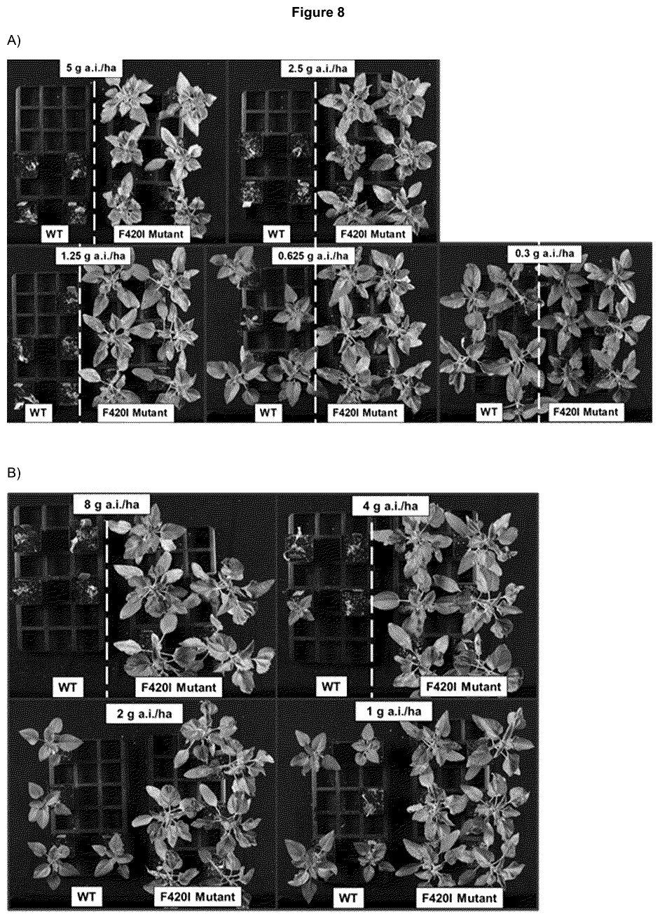

Comparison of the predicted sunflower PPO type II sequence with the Amaranthus sequence shows that the sunflower PPO type II is missing the approximately 30 amino acid N-terminal extension of the Amaranthus PPO type II involved in the additional targeting to the chloroplast. The alignment is shown in FIG. 7.

Thus, the sunflower PPO type II protein would be expected to be targeted only to mitochondria, which in consequence means that mutations in either of the two Sunflower PPO genes would not lead to resistance proteins in both compartments.

### Example 7: Greenhouse PPO Cross-Tolerance Trial

In August of 2021, a greenhouse herbicide trial was carried out in Zwijnaarde, Belgium. A homozygous PPO2_F4201 sunflower seed lot (HA452-M), and a wild type (WT) sunflower line (RHA473) were used in the greenhouse trial. When plants had 2-4 leaves (BBCH12-14 growth stage), they were sprayed with the herbicide treatments indicated in Table 4. Sixteen homozygous PPO2_F4201 mutant plants and six WT plants were sown and treated with each herbicide. Phytotoxicity of mutant and WT plants was recorded at a single time point, 4 days after herbicide treatment (DAT), where 100% phytotoxicity indicates complete dead plants, and 0% phytotoxicity indicates plants without any herbicide injury.

The greenhouse trial had the objective to assess the herbicide tolerance of the mutant and whether PPO2-F4201 mutant individuals survived at the sprayed rates.

TABLE 4
Herbicide treatments used in the greenhouse trial in
Belgium. August 2021. Methylated seed oil adjuvant (MSO)
was added to all herbicide treatments at 1% v/v.

| | Rate |
|---|---|
| Herbicide Treatment | (g a.i./ha) |
| Saflufenacill (800) | 0.3 |
| | 0.625 |
| | 1.25 |
| | 2.5 |
| | 5 |
| Flumioxazin (9155) | 1 |
| | 2 |
| | 4 |
| | 8 |
| Saflufenacil + Trifludimoxazin (851) | 0.625 + 1.25 |
| | 1.25 + 2.5 |
| | 2.5 + 5 |
| | 5 + 10 |
| methyl 2-[2-[2-bromo-4-fluoro-5-[3-methyl-2,6-dioxo-4- | 0.625 |
| (trifluoromethyl)pyrimidin-1-yl]phenoxy]phenoxy]-2- | 1.25 |
| methoxy-acetate (201) | 2.5 |
| | 5 |

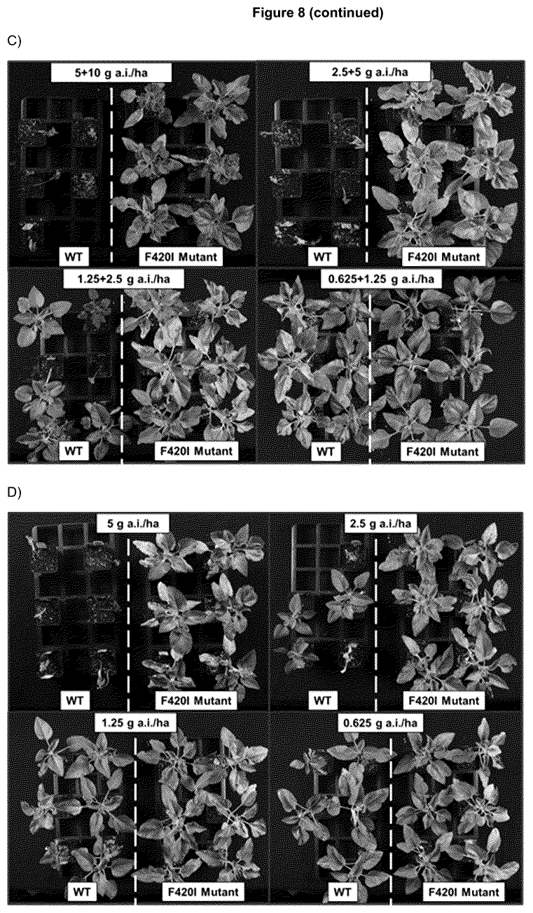

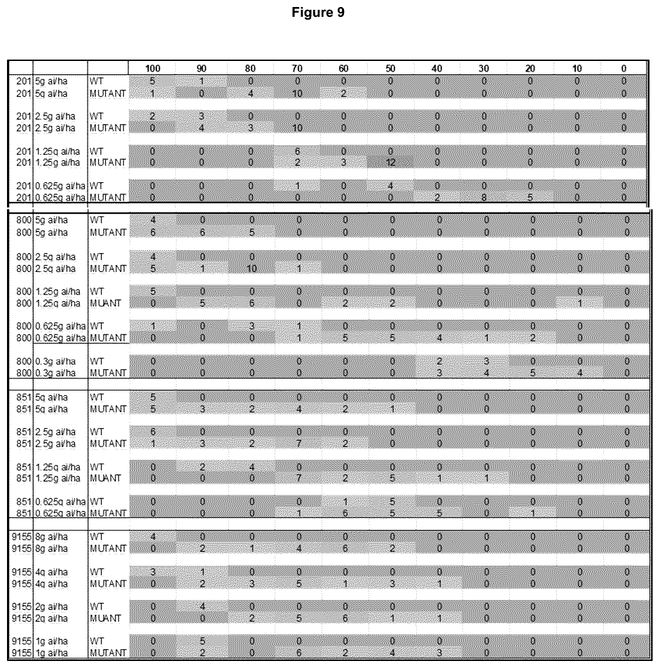

The results are shown in FIGS. 8 and 9.

For all the sprayed herbicides and at all tested rates we observed herbicide tolerance of the mutant, as there was a large difference between herbicide phytotoxicity between the Wild Type and the mutant PPO2_F4201 (FIG. 9 and FIG. 8). Some examples are described below:
WT plants were all killed at a dose as low as 1.25 g/ha of saflufenacil, while all PPO2_F4201 plants survived at all rates, showing some early phytotoxicity at 4 DAT (FIG. 9), followed by consistent survival and recovery at 10 DAT (FIG. 8).
WT plants scored 90-100% at a dose as low as 4 g/ha of flumioxazin with only one surviving WT plant at 10 DAT (FIG. 8), while all PPO2_F4201 plants survived at all rates, showing some early phytotoxicity at 4 DAT (FIG. 9), followed by consistent survival and recovery at 10 DAT (FIG. 8).

As observed in FIG. 8, PPO2_F4201 mutants significantly recover from the phytotoxicity observed at 4 DAT, while WT plants are completely killed since the lowest rate for almost all herbicides. Therefore, the PPO2_F4201 mutation does confer tolerance to sunflower plants carrying the mutation, as opposed to the same sunflower genetic background (WT) without the PPO2_F4201 mutation.

### Example 8: Field PPO Cross-Tolerance Trial

A field trial was carried out to assess PPO cross-tolerance in non-introgressed, selfed, homozygous PPO2_F4201 mutants, as well as to assess tolerance differences using increasing rates of herbicides listed in Table 5. The field trial was done in Utrera, Spain, in September of 2021.

Herbicide treatments (Table 5) were applied in the field when plants had the BBCH12-14 growth stage (2-4 leaves). The field trial design was the randomized complete block design, and 1 replication was considered due to limitation in seed availability. Two entries were used in the field trial, the WT line and the PPO2_F4201 mutant. The experimental plot consisted of 1 entry per plot and 1 row per entry.

Phytotoxicity of mutant and WT entries was recorded over time, at 6, 11 and 14 DAT, where 100% phytotoxicity indicates complete control (dead plants), and 0% phytotoxicity indicates no control or alive plants without any herbicide injury.

TABLE 5
Herbicide treatments used in the greenhouse trial

| Herbicide Treatment | Rate (g a.i./ha) |
|---|---|
| Saflufenacil | 3.125 |
| Saflufenacil | 6.25 |
| Flumioxazin | 45 |
| Flumioxazin | 55 |
| Flumioxazin | 65 |

* all treatments were sprayed with methylated seed oil (MSO) at 1% v/v.

Control of the WT entry was 100% across all saflufenacil rates, while for flumioxazin, WT control was at the 75-80% level across rates (Table 6).

For all herbicides, the PPO2_F4201 mutant clearly showed tolerance when compared to the respective WT background.

Maximum saflufenacil phytotoxicity was 40% in the PPO2_F4201 mutants at 11 and 14 DAT, whereas saflufenacil phytotoxicity in the WT background was 100%. For the PPO2_F4201 mutant, a dose-response between the 2 saflufenacil rates was not observed. Besides, the recorded phytotoxicity is about the same as for higher rates, as indicated in previous field trials results.

For flumioxazin, phytotoxicity in the PPO2_F4201 mutants was only 20% at the highest tested rate of 65 g a.i./ha at 14DAT, while the WT background showed phytotoxicity of 80% at the same evaluation time point.

TABLE 6
Phytotoxicity (%) after application of herbicide treatments in wild type
and PPO2_F420I plants over time in field conditions. Spain, 2021.

| | | Phytotoxicity (%) | | | | | |
|---|---|---|---|---|---|---|---|
| Herbicide | Rate (g | Wild Type (WT) | | | PPO2_F420I Mutant | | |
| Treatment | a.i./ha) | 6DAT | 11DAT | 14DAT | 6DAT | 11DAT | 14DAT |
| Untreated | / | 1 | 1 | 0 | 5 | 6 | 6 |
| Saflufenacil | 3.125 | 100 | 100 | 100 | 40 | 35 | 35 |
| Saflufenacil | 6.25 | 100 | 100 | 100 | 45 | 40 | 40 |
| Flumioxazin | 45 | 80 | 75 | 75 | 20 | 20 | 10 |
| Flumioxazin | 55 | 80 | 70 | 70 | 30 | 25 | 15 |
| Flumioxazin | 65 | 85 | 80 | 80 | 30 | 30 | 20 |

Field analysis showed that the WT entry is completely controlled after treatment with all rates of saflufenacil, while the PPO2_F4201 mutant survives. Wild Type control is at the 75-80% level across flumioxazin rates. It is worth mentioning that the WT is significantly injured at 75-80% even though control is not 100%, while the superior performance of the PPO2_F4201 mutant entry is evident when compared to the WT entry.

### Example 9: Promoter Analysis

### Sunflower Promoter Analysis

In-silico transcript expression analysis studies were performed to assess what influence the promoter would have on PPO gene expression and concurrent herbicide tolerance. Ubiquitin promoters, such as the Ubiquitin3 (UBQ3) and ubiquitin10 (UBQ10) promoter, are widely used to drive constitutive high expression of transgenes (see WO 2012/080975, WO2015/022636 and WO 2016/203377). As a control, the native expression of Arabidopsis UBQ3 and UBQ10 was first compared to the native expression of sunflower UBQ3 and UBQ10 using the software program Genevestigator ((Genevestigator v3: a reference expression database for the meta-analysis of transcriptomes. Tomas Hruz, Oliver Laule, Gabor Szabo, Frans Wessendorp, Stefan Bleuler, Lukas Oertle, Peter Widmayer, Wilhelm Gruissem, Philip Zimmermann, Adv Bioinformatics. 2008; 2008:420747. doi: 10.1155/2008/420747. Epub 2008 Jul. 8. PMID: 19956698; PMCID: PMC2777001). In both sunflower and Arabidopsis, the UBQ10 promoter is about two times as strong as UBQ3 promoter in most tissues (not shown) and also, the level of expression level conferred by both promoters is comparable between the two species.

The expression of the sunflower PPO1 and PPO2 genes driven by their native promoters were compared with the expression of UBQ3 and UBQ10 from sunflower. The in-silico analysis showed that the expression of ppo1 and ppo2 from sunflower is about 15× lower than UBQ3 expression and about 30-50× lower than UBQ10 expression (not shown).

From these analyses, we conclude that the UBQ3 and UBQ10 promoters from Arabidopsis used in many transgenic PPO inhibiting herbicide tolerance drive high expression needed for herbicide tolerance in those instances. However, ppo1 and ppo2 expression in sunflower is clearly lower than UBQ3 and UBQ10. Hence, native expression

1. A non-transgenic sunflower plant comprising a mutated protoporphyrinogen IX oxidase (PPO) gene encoding a mutated sunflower protoporphyrinogen IX oxidase, wherein the mutated sunflower protoporphyrinogen IX oxidase comprises a substitution of phenylalanine (F) to isoleucine (I) at a position corresponding to residue 383 relative to SEQ ID NO: 2 (F383I substitution).

2. The non-transgenic sunflower plant of claim 1, wherein the non-transgenic plant has been obtained by means other than exclusively an essentially biological process.

3. The non-transgenic sunflower plant of claim 1, wherein the mutated protoporphyrinogen IX oxidase comprises: an amino acid sequence as shown in SEQ ID NO: 2, or a variant thereof being at least 98%, or at least 99% or at least 99.5% identical to SEQ ID NO: 2, with the proviso that the variant comprises a substitution of phenylalanine (F) to isoleucine (I) at a position corresponding to residue 383.

4. The non-transgenic sunflower plant of claim 1, wherein the mutated protoporphyrinogen IX oxidase (PPO) gene comprises (a) a nucleic acid sequence as shown in SEQ ID NO: 1, or (b) a nucleic acid sequence being at least 98%, or at least 99% or at least 99.5% identical to SEQ ID NO: 1.

5. The non-transgenic sunflower plant of claim 1, wherein said plant possesses a phenotype of tolerance to one or more PPO-inhibiting herbicides which tolerance is greater than that of a corresponding wild-type sunflower plant.

6. The non-transgenic sunflower plant of claim 1, wherein the mutated PPO gene is present in homozygous form.

7. The non-transgenic sunflower plant of claim 1, wherein the PPO gene has been mutated by EMS (ethyl methanesulfonate) mutagenesis.

8. The non-transgenic sunflower plant of claim 1, wherein the PPO gene has been mutated by radiation induced mutagenesis.

9. The non-transgenic sunflower plant of claim 1, wherein the PPO gene has been mutated by genome editing.

10. The non-transgenic sunflower plant of claim 1, wherein the plant comprises on its leaves an effective amount of one or more PPO-inhibiting herbicides.

11. The non-transgenic sunflower plant of claim 5, wherein the one or more PPO-inhibiting herbicides are selected from the group consisting of: carfentrazone-ethyl, flumioxazin, saflufenacil and/or trifludimoxazin herbicides, and combinations thereof.

12. The non-transgenic sunflower plant of claim 1, wherein the plant comprises a phenotype of tolerance to a level of saflufenacil that would prevent or inhibit the growth of a wild-type plant.

13. The non-transgenic sunflower plant of claim 1, wherein the plant comprises a phenotype of tolerance to saflufenacil that is greater than 80% tolerance to 5 g a.i./ha saflufenacil if applied at a V2-V8 stage.

14. The non-transgenic sunflower plant of claim 1, wherein the gene is operably linked to the native promoter of the protoporphyrinogen IX oxidase (PPO) gene.

15. A seed from the non-transgenic sunflower plant of claim 1, wherein said seed comprises the mutated protoporphyrinogen IX oxidase (PPO) gene.

16. A method for weed control at a plant cultivation site that comprises a non-transgenic sunflower plant of claim 1, comprising applying an effective amount of one or more PPO-inhibiting herbicides at said cultivation site.

17. A method for treating a non-transgenic sunflower plant of claim 1, comprising applying an effective amount of one or more PPO-inhibiting herbicides to said plant.

18. A method for producing sunflower oil comprising (a) growing the non-transgenic sunflower plant of claim 1 at a plant cultivation site, (b) harvesting seeds from the plant, and (c) extracting sunflower oil from the seeds harvested in (b).

19. A method for producing sunflower oil comprising extracting sunflower oil from the seed of claim 15.

20. A method for producing a product from sunflower seeds comprising producing a product from the seed of claim 15.


SEQUENCE LISTING (7 sequences)

SEQ ID NO: 1 - DNA, 1476 bp, synthetic construct
  source 1..1476: mol_type = other DNA
  misc_feature 1..1476: note = Mutated sunflower PPO2 coding sequence (F383I mutation in encoded protein)
atggcttcaccaactacacctgacaaccaaaaacctgctaagcgagttgccgttgttggtgccggtgtcagtggttgtgctgctgcttataagttaaagttacatggattaaatgtcactgtctttgaagctgatgggagagtaggcgggaaactacggagcgtttcgcaagatggtttaatatgggatgagggtgccaatactatgactgagagtgaggcagaggtcagcagtttgattgatgatcttgggcttcgagataaacaacagtttccgatttcacagcacaagcggtacattgtaagaaatggaaaacctgtactgataccttctaacccaattgcactgatcagaagcagttttctttccactcagtcgaaggttcaaattcttttggagccgtttttatggaagaagaataagtcatctgacgcaccagagagtgttggagaattctttcaacgtcattttgggaaggaggttgttgagtatctcattgatccagttgttgcgggaacaagcggcggagatcccgaatcactttctatgcgtcatgcatttcctgagttatgggatttagaaagaaggtttggttcaatcgtttccggggcatttcagtctatggtatcttccagaaatgggaaaaggaaaccttctggaaacagcaaacgtaggcgtggttctttttcgtttttcggtggaatgcagacactcactgatgcactatgcaaagagatcggaccccatgaacttaatcttcaatcaaaggtgcttgaaatgtcctacaactgcgatggtaacgcggttggtaattggtcaatatgttgtgctccagatcaaaacaaacaatttcagcaatctttcgatgctgtgatcatgactgccccacttggcaatgtaaaagaaatgaagattacaaagatcggaagtccgtttccgcttaatttcataccagaggtgagttatatgccaatatcagtaataatctcgactttcaagaaagaaaacgtgaagcagcccctcgagggttttggagttcttgttccagccaaggagcagaaaaatggtttgaggacactaggcactctcttctcctcgatgatgtttccagatcgcgcctcggaagacgtgtacctttacaccaccattgttgggggtagtcgaaacaaagaactggccaaagcttcgagggacgagttgaagcatatagtaacttctgacctcagactgttgcttggaacagaaggggagccaaaatttctaactcactattattggagcaaagcatttccattgtatgggcgcggctatggttcagtcattgaagcaattgagaaaatggaaagggagctacccggatacttttatgcaggtaaccacaaaggagggctctctgtcgggaaagcaatatcctcgggatgcaaagcagccgaatctgtgatcgcatacttggattcttattcggacgagaagtag

SEQ ID NO: 2 - protein, 491 aa, synthetic construct
  REGION 1..491: note = Mutated sunflower PPO2 protein (F383I substitution)
MASPTTPDNQKPAKRVAVVGAGVSGCAAAYKLKLHGLNVTVFEADGRVGGKLRSVSQDGLIWDEGANTMTESEAEVSSLIDDLGLRDKQQFPISQHKRYIVRNGKPVLIPSNPIALIRSSFLSTQSKVQILLEPFLWKKNKSSDAPESVGEFFQRHFGKEVVEYLIDPVVAGTSGGDPESLSMRHAFPELWDLERRFGSIVSGAFQSMVSSRNGKRKPSGNSKRRRGSFSFFGGMQTLTDALCKEIGPHELNLQSKVLEMSYNCDGNAVGNWSICCAPDQNKQFQQSFDAVIMTAPLGNVKEMKITKIGSPFPLNFIPEVSYMPISVIISTFKKENVKQPLEGFGVLVPAKEQKNGLRTLGTLFSSMMFPDRASEDVYLYTTIVGGSRNKELAKASRDELKHIVTSDLRLLLGTEGEPKFLTHYYWSKAFPLYGRGYGSVIEAIEKMERELPGYFYAGNHKGGLSVGKAISSGCKAAESVIAYLDSYSDEK

SEQ ID NO: 3 - DNA, 1476 bp, Helianthus annuus
  source 1..1476: mol_type = unassigned DNA
  misc_feature 1..1476: note = Wild-type sunflower PPO2 (coding sequence)
atggcttcaccaactacacctgacaaccaaaaacctgctaagcgagttgccgttgttggtgccggtgtcagtggttgtgctgctgcttataagttaaagttacatggattaaatgtcactgtctttgaagctgatgggagagtaggcgggaaactacggagcgtttcgcaagatggtttaatatgggatgagggtgccaatactatgactgagagtgaggcagaggtcagcagtttgattgatgatcttgggcttcgagataaacaacagtttccgatttcacagcacaagcggtacattgtaagaaatggaaaacctgtactgataccttctaacccaattgcactgatcagaagcagttttctttccactcagtcgaaggttcaaattcttttggagccgtttttatggaagaagaataagtcatctgacgcaccagagagtgttggagaattctttcaacgtcattttgggaaggaggttgttgagtatctcattgatccagttgttgcgggaacaagcggcggagatcccgaatcactttctatgcgtcatgcatttcctgagttatgggatttagaaagaaggtttggttcaatcgtttccggggcatttcagtctatggtatcttccagaaatgggaaaaggaaaccttctggaaacagcaaacgtaggcgtggttctttttcgtttttcggtggaatgcagacactcactgatgcactatgcaaagagatcggaccccatgaacttaatcttcaatcaaaggtgcttgaaatgtcctacaactgcgatggtaacgcggttggtaattggtcaatatgttgtgctccagatcaaaacaaacaatttcagcaatctttcgatgctgtgatcatgactgccccacttggcaatgtaaaagaaatgaagattacaaagatcggaagtccgtttccgcttaatttcataccagaggtgagttatatgccaatatcagtaataatctcgactttcaagaaagaaaacgtgaagcagcccctcgagggttttggagttcttgttccagccaaggagcagaaaaatggtttgaggacactaggcactctcttctcctcgatgatgtttccagatcgcgcctcggaagacgtgtacctttacaccacctttgttgggggtagtcgaaacaaagaactggccaaagcttcgagggacgagttgaagcatatagtaacttctgacctcagactgttgcttggaacagaaggggagccaaaatttctaactcactattattggagcaaagcatttccattgtatgggcgcggctatggttcagtcattgaagcaattgagaaaatggaaagggagctacccggatacttttatgcaggtaaccacaaaggagggctctctgtcgggaaagcaatatcctcgggatgcaaagcagccgaatctgtgatcgcatacttggattcttattcggacgagaagtag

SEQ ID NO: 4 - protein, 491 aa, Helianthus annuus
  REGION 1..491: note = Wild-type sunflower PPO2 protein
MASPTTPDNQKPAKRVAVVGAGVSGCAAAYKLKLHGLNVTVFEADGRVGGKLRSVSQDGLIWDEGANTMTESEAEVSSLIDDLGLRDKQQFPISQHKRYIVRNGKPVLIPSNPIALIRSSFLSTQSKVQILLEPFLWKKNKSSDAPESVGEFFQRHFGKEVVEYLIDPVVAGTSGGDPESLSMRHAFPELWDLERRFGSIVSGAFQSMVSSRNGKRKPSGNSKRRRGSFSFFGGMQTLTDALCKEIGPHELNLQSKVLEMSYNCDGNAVGNWSICCAPDQNKQFQQSFDAVIMTAPLGNVKEMKITKIGSPFPLNFIPEVSYMPISVIISTFKKENVKQPLEGFGVLVPAKEQKNGLRTLGTLFSSMMFPDRASEDVYLYTTFVGGSRNKELAKASRDELKHIVTSDLRLLLGTEGEPKFLTHYYWSKAFPLYGRGYGSVIEAIEKMERELPGYFYAGNHKGGLSVGKAISSGCKAAESVIAYLDSYSDEK

SEQ ID NO: 5 - DNA, 890 bp, Helianthus annuus
  source 1..890: mol_type = unassigned DNA
  misc_feature 1..890: note = Promoter region of the sunflower PPO2 gene
attcggaagaaaaattagattaccattttatattatttttgttttcattttatattgttttgatattaagttatatgttgttttgtgatctaattctctataaataaaatacattattatataaaacgccaaaaactacaaacaccaaaacgccggtagtgttaaaactgggagaacagctgctgtcaaagcaccttgtaaaaaggggtattaaacgccaaaaaattcactaaaagccaaaaaaaattggtcttctgtaatattatgcaaattttaatgactcgtagtgaattattatacaaaacgccaaaaacgccaaaaaattaaccagaaaacgctataacgccaaaaaattatacatgtgacacaaaaacgattaattcaacgccaaaaagtttaaaagatacaattaacgccaaaaaaaaaacttagacgccataaaatattataccgcgtagggaaaataaaagaagaatgaaaagacaaaaaagcccctccgcccttctctatatcgcgagacacgtgttgggccaggatgcgttctcaccgttctcacacttttgaacgttttatcttgatcgcgtttctatatatatctacccctcaggacccatttttactactcttatagggccttcaagaattcttgaatatacagtaaaaagaatttagaggcacccctgatgttttaggctaaatccgtcactgcttaggatgaactatattagcattatatattaaaattaggtgattaaatcattctataaactaagtgctcaaagcattctataaactaaactacatgttatccttttcagttttagtttatgatatacgtccaaaataataatagctcttgtagtagttaaacccattggtccacagttgga

SEQ ID NO: 6 - protein, 50 aa, Amaranthus tuberculatus
  REGION 1..50: note = First 50 N-terminal amino acids of the Amaranthus tuberculatus PPO2
MVIQSITHLSPNLALPSPLSVSTKNYPVAVMGNISEREEPTSAKRVAVVG

SEQ ID NO: 7 - protein, 20 aa, Helianthus annuus
  REGION 1..20: note = First 20 N-terminal amino acids of the Helianthus annuus PPO2
MASPTTPDNQKPAKRVAVVG